(19) United States
(12) Patent Application Publication
(10) Pub. No.: US 2026/0345563 A1
(43) Pub. Date: Nov. 5, 2026

(54) COMBINATION TREATMENT WITH INHALABLE RAPAMYCIN FORMULATION

(71) Applicant: OrphAI Therapeutics Inc., New Haven, CT (US)

(72) Inventors: Patrick Sarmiere, New Haven, CT (US); Keith Fandrick, New Haven, CT (US); Brigette Roberts, New Haven, CT (US)

(21) Appl. No.: 19/648,816

(22) Filed: Apr. 15, 2026

Related U.S. Application Data
(63) Continuation of application No. PCT/US2026/023634, filed on Apr. 14, 2026.
(60) Provisional application No. 63/789,344, filed on Apr. 15, 2025.

(51) Int. Cl. A61K 31/436; A61K 31/4965; A61K 31/5575; A61K 31/5578; A61K 31/5585; A61P 9/12

(52) U.S. Cl. CPC A61K 31/436; A61K 31/4965; A61K 31/5575; A61K 31/5578; A61K 31/5585; A61P 9/12

(57) ABSTRACT

The present disclosure provides a method for treating a lung disease or disorder in a subject, including administering to the subject a pharmaceutical product, wherein the pharmaceutical product includes an MTOR inhibitor and a prostacyclin or prostacyclin receptor agonist. In some aspects, the product may inhibit mTORC1 activity, inhibit S6 protein phosphorylation, dilate pulmonary vasculature, and/or modulate vascular smooth muscle cell proliferation in the lungs. The product including rapamycin and treprostinil may provide synergistic effects in treating pulmonary hypertension, pulmonary fibrosis, and other chronic lung diseases.

### CROSS-REFERENCE TO RELATED APPLICATIONS

This application is a continuation application of International Application No. PCT/US2026/023634 filed Apr. 14, 2026, now pending; which claims the benefit under 35 USC § 119(e) to U.S. Application Ser. No. 63/789,344 filed Apr. 15, 2025, now pending. The disclosure of each of the prior applications is considered part of and is incorporated by reference in the disclosure of this application.

### INCORPORATION BY REFERENCE OF SEQUENCE LISTING

The material in the accompanying sequence listing is hereby incorporated by reference into this application. The accompanying sequence listing xml file, named AIT1200-1_ST26.xml, was created on Apr. 14, 2026 and is 4,400 bytes in size.

### BACKGROUND OF THE INVENTION

### Field of the Invention

The present invention relates generally to methods and pharmaceutical compositions and products for the treatment of lung diseases, and more specifically to the use of a combination of MTOR inhibitors and prostacyclin, prostacyclin analog, prostanoid or prostacyclin receptor agonists for the prophylaxis and treatment of lung conditions, diseases, and disorders.

### Background Information

Rapamycin was initially discovered as an antifungal metabolite produced by Streptomyces hygroscopicus from a soil sample. Subsequently, rapamycin was found to possess immunosuppressive and anti-proliferative properties in mammalian cells, spurring an interest in identifying the mode of action of rapamycin. Rapamycin was shown to be a potent inhibitor of S6K1 activation, a serine/threonine kinase activated by a variety of agonists and an important mediator of PI3 kinase signaling. Concurrently, the target of rapamycin (TOR) was identified in yeast and animal cells. The mammalian target of rapamycin (mTOR) signaling pathway is a master regulator of cell growth and metabolism. Deregulation of the mTOR pathway has been implicated in a number of human diseases such as cancer, diabetes, obesity, neurological diseases, and genetic disorders. Rapamycin, a specific inhibitor of mTOR, has been shown to be useful in the treatment of certain diseases.

Sirolimus, also known as rapamycin and sold under the brand name Rapamune™ among others, is a macrolide compound that is used to coat coronary stents, prevent organ transplant rejection, treat a rare lung disease called lymphangioleiomyomatosis, and treat perivascular epithelioid cell tumor (PEComa). It has immunosuppressant functions in humans and is especially useful in preventing the rejection of kidney transplants. It is a mammalian target of rapamycin (mTOR) kinase inhibitor that reduces the sensitivity of T cells and B cells to interleukin-2 (IL-2), inhibiting their activity.

Treprostinil is a synthetic prostacyclin analog approved for the treatment of pulmonary arterial hypertension (Group 1) in 2002 and subsequently approved for pulmonary hypertension and pulmonary hypertension-associated interstitial lung disease (Group 3) in 2022. Unlike rapamycin, treprostinil acts primarily as a vasodilator and inhibitor of platelet aggregation through activation of prostacyclin receptors. While treprostinil has been successfully formulated for inhalation delivery, as evidenced by the FDA-approved TYVASO® (United Therapeutics), its mechanism of action differs significantly from rapamycin. Treprostinil does not directly target mTOR signaling but is a potent stimulator of cAMP production which appears to be the primary driver of its pharmacological effects. The resultant elevation in cAMP not only mediates a vasodilatory effect and anti-inflammatory responses but also appears to intersect with the PI3K/Akt/mTOR axis. However, treprostinil's short half-life necessitates frequent dosing, which may limit patient compliance and quality of life. Additionally, systemic side effects such as headache, nausea, and flushing can occur even with inhaled formulations. Thus, while treprostinil supports inhaled therapies for pulmonary hypertension, there remains a need for new treatment options with improved efficacy and reduced dosing frequency.

U.S. Pat. No. 8,492,110 (“the '110 patent”) describes the signaling pathways of the TOR (target of rapamycin) kinase as integrating diverse cellular signals from agents such as nutrients, mitogenic growth factors, energy, and stress-related signals to regulate the catabolic and anabolic processes of the cell. Recent studies have highlighted the significance of TOR kinase in the pathogenesis of lung diseases such as pulmonary hypertension (PH) and pulmonary arterial hypertension (PAH). Complementing the treatment effects of TOR kinase pathways with targeting related pathways offers a synergistic approach to treatment of these lung diseases.

### SUMMARY OF THE INVENTION

The present disclosure is based, in part, upon the development of a safe and effective formulation or pharmaceutical product of a combination of an MTOR inhibitor (e.g., rapamycin) and a prostacyclin (e.g., treprostinil), prostacyclin analog, prostanoid or prostacyclin receptor agonist, that delivers amounts of the active agents to target tissues effective to exert potent biological activity in those target tissues while minimizing toxicity associated with the drugs. In addition, the disclosure describes the pharmacokinetics of a rapamycin composition formulated as described herein, combined with treprostinil. As discussed in more detail infra, the composition and product described herein delivered directly to the lungs will produce markedly higher concentrations of active agents in the lung tissue. Moreover, the amount of active agents in lung tissue achieved by the methods described here is effective to exert potent biological activities including inhibition of cell growth and viability in the target tissue. These biological activities indicate that the delivered dose and/or frequency of rapamycin and treprostinil according to the claimed methods offer improved treatment of lung diseases, including PH and PAH. The formulations described herein are capable of delivering a low, yet therapeutically effective, dose of an MTOR inhibitor (e.g., rapamycin), prostacyclin analog, prostanoid or a prostacyclin (e.g., treprostinil) or prostacyclin receptor agonist), providing for very low systemic exposure to the drugs combined with high efficacy. The result will be an improved therapeutic index for the drugs when administered according to the present disclosure, compared to either active agent alone.

In one embodiment, the disclosure provides a pharmaceutical composition or a product comprising an MTOR inhibitor and a prostacyclin, prostanoid or prostacyclin receptor agonist, or an analog, prodrug, pharmaceutical salt or derivative thereof; and a carrier. In one aspect, the MTOR inhibitor is selected from the group consisting of rapamycin, everolimus, temsirolimus, ridaforolimus, umirolimus, novolimus, myolimus, AP23841, KU-0063794, INK-128, EX2044, EX3855, EX7518, AZD08055, OSI027, zotarolimus, and a rapalog. In one aspect, the prostacyclin, prostacyclin analog, prostanoid or prostacyclin receptor agonist is selected from the group consisting of epoprostenol, iloprost, treprostinil, beraprost, selexipag, treprostinil palmitil inhaled powder (TPIP), long chain treprostinil prodrug, and NS-304. In one aspect, the composition or product includes active agents (e.g., an MTOR inhibitor and a prostacyclin, prostanoid or prostacyclin receptor agonist) formulated for administration by inhalation, oral, intranasal, inhalation, subcutaneous, intravenous, intramuscular, transdermal, buccal, rectal, topical, systemic or parenteral delivery. In another aspect, the composition or product for inhalation is a dry powder formulation. In one aspect, the composition or product comprises 0.1% to 20% w/w treprostinil and 0.1% to 20% rapamycin w/w.

In another embodiment, the disclosure provides a method for treating a disease or disorder in a subject in need thereof comprising administering to the subject a therapeutically effective amount of a composition or product described herein. In one embodiment, the disclosure provides a method for treating a disease or disorder in a subject in need thereof comprising administering to the subject a therapeutically effective amount of a combination treatment of an MTOR inhibitor and a prostacyclin, prostacyclin analog, prostanoid or prostacyclin receptor agonist. In one aspect, administration of the MTOR inhibitor and a prostacyclin, prostacyclin analog, prostanoid or prostacyclin receptor agonist is simultaneously as a single combination formulation, a fixed dose combination (FDC) product or as separate formulations. For example, a fixed dose combination product of a DPI (dry powder inhaler) may include 100 ug rapamycin and 64 ug treprostinil, or for example, 100 ug rapamycin and 100 ug treprostinil. Dosages will be determined by one of skill in the art and will fall within the ranges provided herein (see for example, pmc.ncbi.nlm.nih.gov/articles/PMC2432494/).

In another aspect, administration of the MTOR inhibitor and the prostacyclin, prostacyclin analog, prostanoid or prostacyclin receptor agonist is sequential in either order. In one aspect, administration of the MTOR inhibitor and the prostacyclin, prostacyclin analog, prostanoid or prostacyclin receptor agonist is by inhalation, oral, intranasal, inhalation, subcutaneous, intravenous, intramuscular, transdermal, buccal, rectal, topical, systemic or parenteral delivery. In one aspect, the composition or product for inhalation is a dry powder for pulmonary delivery.

In one aspect, the administration comprises microparticles of the MTOR inhibitor and/or microparticles of the prostacyclin, prostacyclin analog, prostanoid or prostacyclin receptor agonist; particles of a carrier; and optionally at least one excipient, wherein the composition or product is effective to deliver a therapeutic amount of the composition to the lungs. In a preferred aspect, the MTOR inhibitor is rapamycin, and the prostacyclin, prostacyclin analog, prostanoid or prostacyclin receptor agonist is Treprostinil or analogs, derivatives, prodrugs, salts thereof. In one aspect, the rapamycin in the composition or product is from about 0.1% to 20% (w/w). In one aspect, the amount of the treprostinil in the composition or product is from about 0.01% to 20% w/w. In one aspect, the carrier is selected from the group consisting of arabinose, glucose, fructose, ribose, mannose, sucrose, trehalose, lactose, maltose, starches, dextran, mannitol, lysine, leucine, isoleucine, dipalmitoylphosphatidylcholine, lecithin, polylactic acid, poly (lactic-co-glutamic) acid, and xylitol, or mixtures thereof. In certain aspects, the carrier comprises a blend of two different carriers. For example, in one aspect, the carrier comprises a blend of two different lactose carriers.

In certain aspects, the rapamycin and/or treprostinil persist for at least 0.5 to 24 hours after delivery. In one aspect, the composition or product is formulated for once daily, twice daily, three times daily, or four times daily or more administration by any route of administration described herein. For example, the composition or product is formulated for once daily administration and the composition or product comprises rapamycin, treprostinil or a combination thereof. In certain aspects, a total daily dose of rapamycin administered is from 1 ug to 20 mg and in certain aspects, a total daily dose of treprostinil administered is from 1 ug to 20 mg. In certain aspects, the total daily dose of rapamycin administered is from 10 ug to 25 ug and the total daily dose of treprostinil administered is from about 10 to 20 ug, and in one aspect, about 16 ug.

In one aspect, a dose or frequency of treprostinil administered in combination with rapamycin is lower than a dose or frequency of treprostinil administered in the absence of rapamycin. In another aspect, a dose or frequency of rapamycin administered in combination with treprostinil is lower than a dose or frequency of rapamycin administered in the absence of treprostinil.

Optionally, the method includes administering one or more additional therapeutic agents to the subject, e.g., a different MTOR inhibitor or a different prostacyclin or prostacyclin receptor agonist, or unrelated drug.

It is believed that the methods described herein result in one or more of inhibiting the biological activity of mTORC1 in the lung; inhibiting phosphorylation of the S6 protein in the lung, inhibiting platelet aggregation, dilates pulmonary and systemic arterial vascular beds, or inhibits smooth muscle cell proliferation; and/or interfering with the cell cycle progression of vascular smooth muscle cells (VSMCs) by elevating expression of inhibitor of DNA binding 1 (ID1). Additionally, mTORC1 inhibition may modulate pulmonary artery endothelial cell behavior by reducing endothelial cell dysfunction, attenuating aberrant proliferation and migration, and restoring angiogenic balance, thereby contributing to improved vascular homeostasis and mitigating pulmonary vascular remodeling.

In one aspect, the disease or disorder is a systemic disease or disorder or a lung disease or disorder. For example, a systemic disease or disorder is systemic sclerosis or scleroderma. Another example is wherein the lung disease or disorder is pulmonary hypertension, idiopathic pulmonary fibrosis, progressive pulmonary fibrosis, right heart failure secondary to pulmonary hypertension, lymphangioleiomyomatosis, COPD, chronic fibrotic lung disease, interstitial lung disease, chronic lung disease, pulmonary capillary hemangiomatosis, or hereditary hemorrhagic telangiectasia, idiopathic interstitial pneumonia, pulmonary sarcoidosis, systemic sclerosis associated ILD, fibrotic lung disorders, chronic hypersensitivity pneumonitis (CHP), occupational lung disease (drug or radiation-induced) or combined pulmonary fibrosis and emphysema (CPFE), Pulmonary hypertension associated with COPD, idiopathic pulmonary hypertension, pulmonary hypertension due to lung disease and/or hypoxia, Group 5 PH due to unclear or multifactorial etiology, hematologic disorders, myeloproliferative disorders, sarcoidosis, Langerhans cell histiocytosis, LAM, neurofibromatosis, vasculitis, metabolic diseases, glycogen storage diseases, Gaucher disease, or thyroid disorders. For example, the pulmonary hypertension is pulmonary arterial hypertension, pulmonary hypertension associated with interstitial lung disease, chronic thromboembolic pulmonary hypertension, or sarcoidosis-associated pulmonary hypertension.

In another embodiment, the disclosure provides a method of treating a subject with pulmonary hypertension comprising administering by inhalation a dose and frequency of rapamycin at about 100 ug at least once daily and administering treprostinil by inhalation or orally at a dose of 0.25 mg to 1 mg at least twice daily. In one aspect the inhalation dose is administered using a dry powder inhaler.

Dosing and frequency of dosing, including modes of administration will be determined by a healthcare professional and are understood from the present disclosure and prior approved and studied MTOR inhibitors and prostacyclin, prostacyclin analog, prostanoid and prostacyclin receptor agonists. For example, illustrative examples include administration of rapamycin is once daily (QD) or twice daily (BID); administration of treprostinil is QD or BID; administration of rapamycin is QD or BID and administration of treprostinil is greater than BID (e.g., administer rapamycin alone or in combination with Treprostinil once or twice followed by additional administration of treprostinal another 1-2 times in a day). Treprostinal may therefore be administered up to about three or four times daily, however, it is possible that the dose of Treprostinil is reduced when provided on a day when rapamycin is also administered. In one aspect, the dosage of rapamycin and/or treprostinil is the same at each administration, yet, in another aspect, the dosage of rapamycin and/or treprostinil varies with administrations.

In one embodiment, the disclosure provides a pharmaceutical composition for inhaled, subcutaneous, intravenous or oral administration comprising a combination of rapamycin and treprostinil, wherein the rapamycin is administered DPI and is present in an amount of about 0.025 mg to 3.0 mg and treprostinil is present in an amount of about 18 to 64 ug for inhalation, about 0.5-2.5 ng/kg/min for subQ or IV, and about 0.125 to 0.5 mg for oral administration. In one aspect, the rapamycin is present in an amount of about 0.3 mg to about 2.0 mg. In one aspect, the rapamycin is present in an amount of up to about 2.0 mg. In another aspect, the amount of rapamycin and/or the amount of treprostinil is less than about 1 mg. In one aspect, the composition is formulated for oral administration in a form selected from a tablet, a caplet, a capsule, a liquid, or a dry powder.

In another embodiment, the disclosure provides a pharmaceutical product including (i) rapamycin, an analog thereof, a prodrug thereof or a derivative thereof, formulated as a dry powder for inhalation, (ii) treprostinil, an analog thereof, a prodrug thereof or a derivative thereof, formulated for inhalation, oral administration, intravenous (IV) administration or subcutaneous administration for simultaneous, separate or sequential administration. In one aspect, the rapamycin, analog thereof, prodrug thereof or derivative thereof is selected from rapamycin, everolimus, temsirolimus, ridaforolimus, umirolimus, novolimus, myolimus, AP23841, KU-0063794, INK-128, EX2044, EX3855, EX7518, AZD08055, OSI027, zotarolimus, or a rapalog. In another aspect, the treprostinil analog thereof, prodrug thereof or derivative thereof is selected from epoprostenol, iloprost, treprostinil, treprostinil sodium, treprostinil diethanolamine, treprostinil diolamine, treprostinil palmitil, treprostinil phosphate, treprostinil diproprionate, treprostinil diacetate, treprostinil dibutanoate, beraprost, selexipag, or NS-304. In another aspect, the product includes 0.1% to 20% w/w treprostinil and 0.1% to 20% rapamycin w/w. In another aspect, the rapamycin and/or the treprostinil further include a pharmaceutically acceptable carrier, optionally selected from the group consisting of arabinose, glucose, fructose, ribose, mannose, sucrose, trehalose, lactose, maltose, starches, dextran, mannitol, lysine, leucine, isoleucine, dipalmitoylphosphatidylcholine, lecithin, polylactic acid, poly(lactic-co-glutamic) acid, and xylitol, or mixtures thereof. In some aspects, the pharmaceutically acceptable carrier comprises a blend of two different carriers. In various aspects, the pharmaceutically acceptable carrier comprises a blend of two different lactose carriers. In one aspect, the rapamycin is present in an amount of about 0.025 mg to 3.0 mg. In another aspect, the treprostinil is present in an amount of about 18 to 64 ug for inhalation, about 0.5 to 2.5 ng/kg/min for subcutaneous or IV administration, and about 0.125 to 0.5 mg for oral administration. In one aspect, the rapamycin is present in an amount of about 0.3 mg to about 2.0 mg. In another aspect, the rapamycin is present in an amount of up to about 2.0 mg. In some aspects, the amount of rapamycin is less than about 1 mg. In other aspects, the amount of treprostinil is less than about 1 mg.

In one aspect, the composition is administered at a total daily dose of rapamycin administered is about 100 ug and the total daily dose of treprostinil is about 128 ug. In one aspect, the composition is administered at a total daily dose of rapamycin is administered in a total daily dose of 200 ug and treprostinil is administered in a total daily dose of about 128 ug. In one aspect, the rapamycin is administered BID. In another aspect, the treprostinil is administered in an amount that is greater than 128 ug. In one aspect, the rapamycin is administered in a total daily dose of about 200 ug and treprostinil is administered in a total daily dose of greater than about 128 ug.

It is understood that the dosage and frequency of rapamycin and treprostinil administration may be predetermined in a labeled package. For example, the package is a blister pack, a vial, or a metered inhalation device. In one aspect, treprostinil is available in single-dose cartridges containing 16 ug, 32 ug, 48 ug, or 64 ug of treprostinil. (Note: mcg, ug or ug may be used interchangeably herein to reflect microgram).

### BRIEF DESCRIPTION OF THE DRAWINGS

FIGS. 1A-1C illustrate highest Single Agent (HSA) synergy scores for Treprostinil and Rapamycin in Human PASMCs. FIG. 1A shows a mean HSA synergy (S_{HSA}) matrix across all donors and replicates for combinations of treprostinil (2-10 nM) with rapamycin (0.1 or 0.25 nM) after 48 h of treatment in PDGF-stimulated hPASMCs. FIG. 1B illustrates donor-level HSA scores across three independent hPASMC donors with 0.1 nM rapamycin. FIG. 1C illustrates donor-level HSA scores across three independent hPASMC donors with 0.25 nM rapamycin. Scores >10 are considered strong biologically meaningful synergy, scores between −10 and 10 were likely additive, and scores <−10 were considered antagonistic. Scales reflect the range of interaction scores within each panel, with darker shading corresponding to stronger synergy.

FIGS. 2A-2C illustrate Bliss Synergy Scores for Treprostinil and Rapamycin Combinations in Human PASMCs. FIG. 2A shows a synergy matrix showing mean Bliss Synergy Scores (SBliss) across all donors and replicates for combinations of treprostinil (2-10 nM) with rapamycin (0.1 or 0.25 nM) after 48 h treatment in PDGF-stimulated hPASMCs. FIG. 2B illustrates donor-level Bliss scores across three independent hPASMC donors for 0.1 nM rapamycin. FIG. 2C illustrates donor-level Bliss scores across three independent hPASMC donors for 0.25 nM rapamycin Values >10 indicate strong synergy, values between −10 and 10 are likely additive, and <−10 values indicate antagonism. Scales reflect the range of interaction scores in each panel, with darker shades representing stronger synergy.

### DETAILED DESCRIPTION OF THE INVENTION

The present invention provides methods and compositions for the treatment and prophylaxis of lung diseases and disorders in a subject in need of such treatment. In one embodiment, the methods include administering to the subject via inhalation a composition including rapamycin and treprostinil in a suitable carrier, and optionally one or more additives.

The term “rapamycin” is used generically throughout this invention disclosure to refer to rapamycin itself (also referred to as sirolimus) as well as to its prodrugs (such as temsirolimus) and prodrugs, rapalogs, and derivatives. Derivatives of rapamycin include compounds that are structurally similar to rapamycin, are in the same chemical class, are rapamycin analogs, or are pharmaceutically acceptable salts of rapamycin or its derivatives. Further description and examples of rapamycin, its prodrugs, and derivatives are provided in the following section.

The term “treprostinil” is used generically to refer to treprostinil as well as compounds that are structurally similar or are in the same chemical class. Similar compounds include treprostinil analogs, or pharmaceutically acceptable salts of Treprostinil, prodrugs (e.g., Treprostinil palmitil) or its derivatives. Similar compounds can include prostaglandin analogs such as epoprostenol, beraprost, and iloprost. Treprostinil analogs can include compounds such as treprostinil diethanolamine, treprostinil diolamine, treprostinil palmitil, treprostinil phosphate, treprostinil diproprionate, treprostinil diacetate, treprostinil palmitil inhaled powder (TPIP), long chain treprostinil prodrug (C12, C14 or C16 long chain treprostinil prodrug), or treprostinil dibutanoate.

The anti-inflammatory and anti-proliferative effects of both rapamycin and treprostinil can work synergistically to slow disease progression. Treprostinil's vasodilatory effects can be complemented by rapamycin's ability to prevent vascular remodeling. The present disclosure shows that, surprisingly, the combination of treprostinil and rapamycin can address both the functional and structural abnormalities in the pulmonary arteries, offering a comprehensive treatment strategy for lung diseases and disorders such as pulmonary hypertension (PH) and pulmonary arterial hypertension (PAH). (see Tables 1, 2 and 3): See Wexler, M., 2022, pulmonaryhypertension news.com/news/pah-patients-prefer-yutrepia-over-tyvaso-inspire-study/; Carvalho, J. 2021 pulmonaryhypertensionnews.com/news/insmed-pah-therapy-tpip-phase-1-trial-results/; Ismat, F. A. et al., 2022 Advances in Therapy 39:5144-5157; Liquidia Corporation, 2022, www.globenewswire.com/news-release/2022/08/01/248931I/O/en/Liquidia-Announces-the-Publication-of-Long-Term-Clinical-Data-from-Completed-INSPIRE-Study-in-the-Journal-Pulmonary-Circulation.html.

TABLE 1
Broad Cardiopulmonary Disease Considerations for Rapamycin and Treprostinil

| Indication | Rationale for Combination Therapy |
|---|---|
| Pulmonary Arterial | Treprostinil provides vasodilation and anti- |
| Hypertension | remodeling effects, while rapamycin inhibits |
| (PAH) | vascular smooth muscle proliferation. |
| Pulmonary Hypertension | Treprostinil improves pulmonary hemodynamics, |
| with Interstitial Lung | and rapamycin may reduce fibrosis and |
| Disease (PH-ILD) | inflammation. |
| Idiopathic Pulmonary | Treprostinil enhances oxygenation and exercise |
| Fibrosis (IPF) | capacity, while rapamycin targets fibrotic |
| | pathways. |
| Chronic Thromboembolic | Treprostinil improves blood flow, and rapamycin |
| Pulmonary Hypertension | may prevent vascular remodeling and thrombosis. |
| (CTEPH) | |
| Right Heart Failure | Treprostinil reduces pulmonary vascular resistance, |
| Secondary to PH | and rapamycin may protect against cardiac hypertrophy. |
| Lymphangioleiomyomatosis | Treprostinil improves oxygenation, while rapamycin |
| (LAM) | targets mTOR pathway dysregulation in LAM cells. |
| Pulmonary Capillary | Treprostinil enhances oxygenation, while rapamycin |
| Hemangiomatosis (PCH) | may inhibit abnormal capillary proliferation. |
| Sarcoidosis-Associated | Treprostinil improves pulmonary hemodynamics, and |
| Pulmonary Hypertension | rapamycin may modulate granulomatous inflammation. |
| Hereditary Hemorrhagic | Treprostinil improves blood flow, and rapamycin may |
| Telangiectasia (HHT) | reduce vascular malformations. |

www.uptodate.com/contents/treatment-of-pulmonary-arterial-hypertension-group-1-in-adults-pulmonary-hypertension-specific-therapy; link.springer.com/article/10.1007/s13665-023-00311-2.

In one aspect, the disease or disorder is a systemic disease or disorder or a lung disease or disorder. For example, a systemic disease or disorder is systemic sclerosis or scleroderma. Another example is wherein the lung disease or disorder is pulmonary hypertension, idiopathic pulmonary fibrosis, progressive pulmonary fibrosis, right heart failure secondary to pulmonary hypertension, lymphangioleiomyomatosis, COPD, chronic fibrotic lung disease, interstitial lung disease, chronic lung disease, pulmonary capillary hemangiomatosis, or hereditary hemorrhagic telangiectasia, idiopathic interstitial pneumonia, pulmonary sarcoidosis, systemic sclerosis associated ILD, fibrotic lung disorders, chronic hypersensitivity pneumonitis (CHP), occupational lung disease (drug or radiation-induced) or combined pulmonary fibrosis and emphysema (CPFE), Pulmonary hypertension associated with COPD, idiopathic pulmonary hypertension, pulmonary hypertension due to lung disease and/or hypoxia, Group 5 PH due to unclear or multifactorial etiology, hematologic disorders, myeloproliferative disorders, sarcoidosis, Langerhans cell histiocytosis, LAM, neurofibromatosis, vasculitis, metabolic diseases, glycogen storage diseases, Gaucher disease, or thyroid disorders. For example, the pulmonary hypertension is pulmonary arterial hypertension, pulmonary hypertension associated with interstitial lung disease, chronic thromboembolic pulmonary hypertension, or sarcoidosis-associated pulmonary hypertension.

The inhalable compositions and products described herein are referred to as “aerosol formulations” and are meant to describe aerosolizable compositions suitable for producing respirable particles or droplets containing a combination formulation, which as described above refers to a combination formulation including rapamycin itself, preferably in the amorphous form described as sirolimus, or an analog thereof. In one aspect, the combination formulation also includes a treprostinil compound or an analog thereof. In some aspects, the rapamycin is selected from sirolimus, everolimus, temsirolimus, ridaforolimus, umirolimus, novolimus, myolimus, AP23841, KU-0063794, INK-128, EX2044, EX3855, EX7518, AZD08055, OSI027, zotarolimus, or a rapalog, and the treprostinil compound is selected from treprostinil, treprostinil sodium, treprostinil diethanolamine, treprostinil diolamine, treprostinil palmitil, treprostinil phosphate, treprostinil diproprionate, treprostinil diacetate, treprostinil dibutanoate, epoprostenol, iloprost, treprostinil, beraprost, selexipag, treprostinil palmitil inhaled powder (TPIP), long chain treprostinil prodrug, or NS-304. In one aspect, the rapamycin is sirolimus. See for example, U.S. Pat. No. 11,491,143 for inhalable rapamycin formulations, herein incorporated by reference in its entirety.

In one embodiment, the present disclosure provides a method for treating a lung disease or disorder in a subject in need thereof, the method includes administering to the subject a therapeutically effective amount of rapamycin and treprostinil, or analogs thereof. In some aspects, administration is simultaneously in a single composition, a fixed dose combination product or in separate compositions. In some aspects, administration of rapamycin and treprostinil is sequential. The MTOR inhibitor and the prostacyclin, prostacyclin analog, prostanoid, or prostacyclin receptor agonist can be administered in a fixed dose combination product or in variable doses. In some aspects, administration is by inhalation or by oral delivery. In some aspects, the composition or product for inhalation is a dry powder for pulmonary delivery.

The aerosol formulations described herein may include a combination formulation with both rapamycin and treprostinil, or only rapamycin or treprostinil. The aerosol formulations include a carrier, and optionally one or more additives. The aerosol formulations may be in the form of an aqueous solution, a dry powder, or a mixture of one or more pharmaceutically acceptable propellants and a carrier, as described in detail in the section below.

The oral formulations described herein may include a combination formulation with both rapamycin and treprostinil, or only rapamycin or treprostinil. The oral formulations may be in the form of a tablet, capsule, caplet, or liquid, and include one or more pharmaceutically acceptable carriers. In some aspects, the oral formulation is rapamycin, and the inhalable aerosol formulation is treprostinil. In some aspects, the oral formulation is treprostinil, and the inhalable aerosol formulation is rapamycin.

The terms “administration of” and or “administering” should be understood to mean providing a pharmaceutical composition in a therapeutically effective amount to the subject in need of treatment. Administration routes can be intranasal through inhalation, oral, parenteral, or topical; however, the compositions described herein are preferably inhaled or delivered orally.

As used herein, the term “product” or “pharmaceutical product” is meant to refer to the combination of two active agents or drugs such as (i) rapamycin, an analog thereof, a prodrug thereof or a derivative thereof, and (ii) treprostinil, an analog thereof, a prodrug thereof or a derivative thereof, that are not necessarily physically mixed in the same pill or vial. Both active agents are meant to be used together, because of the synergistic effects associated with said combination, but the focus is meant to be on the functional coupling rather than the physical proximity. That is, the active agents are intended to be used together, regardless of the delivery timing or vessel. The active agents can be administered simultaneously, in a particular sequence, or completely separately. Additionally, the active agents can be administered by a same route of administration or via a different route of administration. It is to be understood that when referring to the administration of the active agents simultaneously, and using a same route of administration, the pharmaceutical product can be referred to as a pharmaceutical composition, as the active agents may be delivered as a single therapy (e.g., injection, inhalation, pill, . . . ). Unless explicitly described otherwise, the term composition (which generally refers to the combination of the active agents in a same formulation, may include any combination of the active agents (in different formulation and/or for different administration route, as generally referred to as a product).

The pharmaceutical compositions and products can be administered in a variety of unit dosage forms depending upon the method of administration. Suitable unit dosage forms include, but are not limited to, powders, nasal sprays, nebulizable solutions, capsules, tablets, syrups, suspensions, lozenges, elixirs, or granules.

In some aspects, administration can be in combination with one or more additional therapeutic agents. The phrases “combination therapy,” “combined with,” and the like refer to the use of more than one medication or treatment simultaneously to increase the response. The composition and products of the present disclosure might, for example, be used in combination with other drugs or treatments to treat lung diseases.

While not wanting to be bound by a particular theory, it is believed that the combination formulation, product or delivery described herein has several advantages over the existing monotherapies, for example: 1) Enhance Vasodilation: Treprostinil's vasodilatory effects can be complemented by rapamycin's ability to prevent vascular remodeling; 2) Reduce Inflammation and Proliferation: The anti-inflammatory and anti-proliferative effects of both drugs can work synergistically to slow disease progression; and 3) Improve Overall Vascular Health: Together, they can address both the functional and structural abnormalities in the pulmonary arteries, offering a comprehensive treatment strategy for PH.

In the context of the pharmaceutical compositions and products of the disclosure, a “carrier” refers to, for example, a liquid or solid material such as a solvent, a diluent, stabilizer, adjuvant, excipient, auxiliary agent, propellant, or vehicle with which rapamycin or treprostinil is formulated for delivery. Examples of pharmaceutically acceptable carriers for use in the compositions and products of the disclosure include, without limitation, dry powder carriers such as lactose, mannose, amino acids, cyclodextrin, dipalmitoylphosphatidylcholine, hydrocarbon and fluorocarbon propellants, compressed gases, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof. Preferably, in the context of the dry powder aerosol formulations of rapamycin, treprostinil, or a combination thereof, the carrier, if present, is selected from the group consisting of a saccharide and a sugar alcohol. In one aspect, the carrier, if present, is lactose. In the context of oral formulations, in one aspect, the carrier is a binder, a lubricant, a preservative, an antioxidant, a disintegrating agent, a thickener, a buffer, a surfactant, a suspending agent, an emulsifying agent, a solvent, a stabilizer, or a bioavailability modifier.

In some aspects, the composition or product includes pharmaceutically acceptable carriers, excipients, or stabilizers. In some aspects, the carriers, excipients or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight peptides (less than about 10 amino acid residues); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrin; chelating agents such as EDTA; sugars such as sucrose, mannitol, dextrin, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carriers include, but are not limited to, liposomes, nanoparticles, ointments, micelles, microspheres, microparticles, and emulsions. Examples of excipients include, but are not limited to, anti-adherents such as magnesium stearate, binders such as saccharides and their derivatives (sucrose, lactose, starches, cellulose, sugar alcohols, and the like), proteins such as gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate, and parabens. Examples of diluents include, but are not limited to, water, alcohol, saline solution, glycol, mineral oil, and dimethyl sulfoxide (DMSO). In further aspects, the pharmaceutical compositions include one or more additional therapeutically or biologically active substances. In some aspects, the rapamycin and the treprostinil persist for at least 12 or 24 hours after delivery. In some aspects, a lung to blood concentration ratio of the rapamycin at 12 or 24 hours after delivery is at least 100, at least 250, or at least 500. In some aspects, a lung to blood concentration ratio of the treprostinil at 12 or 24 hours after delivery is at least 20, at least 50, at least 100, at least 250, or at least 500.

In one aspect, the molar ratio of rapamycin or MTOR inhibitor to Treprostinil or prostacyclin, prostacyclin analog, prostanoid/prostacyclin receptor agonist is in the range of about 20:1 to 1:20; about 20:1 to 3:1; about 20:1 to 4:1; about 20:1 to 5:1; about 15:1 to 1:1, about 15:1 to 3:1; about 15:1 to 4:1; about 15:1 to 5:1; about 10:1 to 3:1; about 10:1 to about 4:1; about 10:1 to about 5:1; about 5:1 to about 4:1; about 5:1 to about 3:1; or about 5:1 to 1:1 and from about 1:20, about 1:15, about 1:10, 1:5, 1:4, 1:3, 1:2 or any combination thereof.

In one aspect, the administered dose of the composition or product is sufficient to achieve therapeutic levels of rapamycin and treprostinil in the lung tissue while maintaining a low blood level, or a low blood trough level, in the subject. For example, the therapeutic levels of rapamycin may be from about 1 ng/g to about 50 ng/g, about 1 ng/g, about 5 ng/g, about 10 ng/g, about 15 ng/g, about 20 ng/g, about 25 ng/g, about 50 ng/g and the blood trough level is from 0.01 to 0.15 ng/ml, from 0.075 to 0.350 ng/mL, from 0.150 to 0.750 ng/mL, from 0.750 to 1.5 ng/mL, or from 1.5 to 5 ng/mL. In one aspect, the administered dose is sufficient to achieve a therapeutic level of rapamycin in the lung of from about 5 ng/g to 50 ng/g, or from about 5 ng/g to 20 ng/g, and a blood trough level of drug of less than 5 ng/ml, less than 2 ng/ml, less than 1 ng/ml, or less than 0.5 ng/mL. In one embodiment, the lung to blood concentration ratio of the rapamycin composition 24 hours after delivery is at least 10, at least 50, at least 100, at least 250, or at least 500. In one embodiment, the lung to blood concentration ratio of the rapamycin composition 24 hours after delivery is from about 10 to 50, 50 to 100, 100 to 250, 250 to 500, 500 to 750, or 750 to 1000.

The method also delivers an amount of treprostinil effective to achieve a maximum blood level from about 0.01 ng/mL to 10 ng/ml, 0.1 to 0.5 ng/ml, 0.5 to 1.5 ng/mL, 1.5 to 2.5 ng/ml, 2.5 to 3.5 ng/mL, 3.5 to 4.5 ng/mL, 0.2 ng/mL, 0.5 ng/mL, 1 ng/mL, 2 ng/ml, 3 ng/mL, 4 ng/ml, or 5 ng/mL, and the blood trough level is from 0.01 to 5 ng/mL, from 0.075 to 1 ng/mL, from 1 to 2 ng/ml, from 2 to 3 ng/ml, or from 3 to 5 ng/mL. In one embodiment, the lung to blood concentration ratio of the treprostinil composition 24 hours after delivery is at least 100, at least 250, or at least 500. In one embodiment, the lung to blood concentration ratio of the treprostinil 24 hours after delivery is at least 100, at least 250, or at least 500. In one embodiment, the lung to blood concentration ratio of the treprostinil 24 hours after delivery is from about 100 to 250, 250 to 500, 500 to 750, or 750 to 1000. Preferably, the aforementioned therapeutic levels are achieved by administering an aerosol formulation described herein once a day.

In one embodiment, the total daily dose of rapamycin is in the range of from 5 to 100 micrograms, from 20 to 250 micrograms, from 50 to 500 micrograms (0.05 to 0.5 milligrams), from 250 to 1000 micrograms (0.25 to 1 milligrams) or from 500 to 2000 micrograms (0.5 to 2 milligrams). In one embodiment, the total daily dose is less than 500 micrograms, less than 100 micrograms, less than 50 micrograms, less than 20 micrograms, or less than 10 micrograms. In one embodiment, the total daily dose is less than 500 micrograms, less than 250 micrograms, less than 100 micrograms, less than 50 micrograms, or less than 10 micrograms. In one embodiment, the total daily dose administered to the subject is less than 0.5 mg or less than 0.25 mg per day. For oral rapamycin, doses are in the range of 0.05 to 15 mg. Further aspects of delivery and dosing, including combination therapies, are described below.

In some aspects, a total daily dose of treprostinil is from about 10 ug to about 200 ug. In some aspects, the total daily dose is less than 500 ug, less than 100 ug, less than 50 ug, less than 20 ug, or less than 10 ug of treprostinil. In some aspects, the total daily dose of treprostinil administered to the subject is less than 0.5 mg or less than 0.25 mg per day.

As used herein, the effective amount of a composition or product of the present disclosure refers to the amount sufficient to reduce or ameliorate the progression, severity, and/or duration of a disease or disorder, or one or more symptoms of a disease or disorder, to prevent the advancement of a disease or disorder, cause the regression of a disease or disorder, to prevent the development or onset of one or more symptoms associated with a disease or disorder, to enhance or improve the prophylactic or therapeutic effect(s) of another therapy (e.g., a prophylactic or therapeutic agent) with respect to the severity or onset of one or more symptoms of a disease or disorder, or with respect to the development or progression of a disease or disorder. Thus, in the context of the methods of the invention, the terms “treat,” “treatment,” and “treating” refer to the reduction of the severity, duration, or progression of a disease or disorder or one or more symptoms associated with a disease or disorder.

In certain aspects, the methods include pulmonary administration of a composition or product of the present disclosure as the primary therapy. In other aspects, the administration of a composition or product of the disclosure is an adjuvant therapy. In either case, the methods provided herein contemplate the administration of a composition or product of the disclosure in combination with one or more additional therapies for the treatment of a disease or disorder. The terms “therapy” and “therapies” refer to any method, protocol, and/or agent that can be used in the prevention, treatment, management, or amelioration of a disease or disorder or one or more symptoms thereof.

The one or more additional therapies may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a composition of the invention.

In some aspects, an additional therapeutic agent is formulated for co-administration with a composition or product of the disclosure in a dosage form for pulmonary administration. In other aspects, an additional therapeutic agent is administered as a fixed dose combination product or separately from the dosage form that contains the composition of the invention and by the same or different route of administration as the composition or product of the disclosure. The methods of the disclosure also contemplate a combination of one or more additional therapeutic agents for administration concomitantly with, before, or after the administration of the dosage form including the composition or product of the disclosure.

In certain aspects, the methods of the disclosure are effective to manage a disease or disorder in a subject having the disease or disorder. In this context, the terms “manage,” “managing,” and “management” refer to the beneficial effects that a subject derives from a therapy that does not result in a cure. In one aspect, the disease or disorder is managed in the subject if its progression is slowed or stopped during treatment with rapamycin according to the methods of the disclosure. In another aspect, the disease or disorder is managed in the subject if one or more symptoms associated with the disease or disorder are ameliorated or stabilized (i.e., the symptom does not worsen during the course of treatment).

In one aspect, the methods of the disclosure are directed to subjects who are “non-responsive” or “refractory” to a currently available therapy for the disease or disorder. In this context, the terms “non-responsive” and “refractory” refer to the subject's response to therapy as not clinically adequate to relieve one or more symptoms associated with the disease or disorder. The terms “subject” and “patient” are used interchangeably in this disclosure. The terms refer to an animal, preferably a mammal, including a non-primate (e.g., a cow, pig, horse, cat, dog, rat, or mouse) and a primate (e.g., a chimpanzee, a monkey such as a cynomolgus monkey or a human), and more preferably a human. In a preferred aspect, the subject is a human.

The terms “prevent”, “preventing”, and “prevention” refer to the prevention of the recurrence, development, progression, or onset of one or more symptoms of the disease or disorder resulting from the administration of one or more compounds identified in accordance the methods of the disclosure or the administration of a combination of such a compound and a known therapy for a disease or disorder.

Preferably, the administration of a composition or product according to the methods of the disclosure in combination with one or more additional therapies provides a synergistic response in the subject having a disease or disorder. In this context, the term “synergistic” refers to the efficacy of the combination being more effective than the additive effects of either single therapy alone. In one aspect, the synergistic effect of rapamycin and treprostinil therapy, according to the disclosure, permits the use of lower dosages and/or less frequent administration of at least one therapy in the combination compared to its dose and/or frequency outside of the combination. In another aspect, the synergistic effect is manifested in the avoidance or reduction of adverse or unwanted side effects associated with the use of either therapy alone.

The term “pharmaceutically acceptable” indicates approval by a regulatory agency of the Federal or a state government or listed in the U.S. Pharmacopeia or other generally recognized pharmacopeia, such as the European Pharmacopeia, for use in animals, and more particularly in humans. One method for poorly solubilizing water soluble or water insoluble drugs is to form a salt of the drug or to prepare a prodrug that is more soluble itself, or that can be used to form a water-soluble salt of the prodrug. Methods for forming salts and pharmaceutically acceptable salt forms are known in the art and include, without limitation, salts of acidic or basic groups that may be present in the drug or prodrug of interest. Compounds that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

In one aspect, the methods, products, and compositions of the disclosure utilize a prodrug, salt, analog or derivative of rapamycin or treprostinil, preferably temsirolimus or related compound. In one aspect, the methods, products, and compositions of the disclosure utilize rapamycin (sirolimus).

### Rapamycin

Rapamycin is a macrocyclic lactone produced by Streptomyces hygroscopicus. Its chemical (IUPAC) name is (3S,6R,7E,9R,10R,12R,14S,15E,17E,19E,21S,23S,26R, 27R,34aS)-9,10,12,13,-14,21,22,23,24,25,26,27,32,33,34,34a-hexadecahydro-9,27-dihydroxy-3-[(1R)-2-[(1S,3R,4R)-4-hydroxy-3-methoxy cyclohexyl]-1-methylethyl]-10,21-dimethox-y-6,8,12, 14,20,26-hexamethyl-23,27-epoxy-3H-pyrido[2,1-c][1,4]oxaazacyclohentriacontine-1,5, 11,28,29 (4H,6H,31H)-pentone.

Its molecular formula is C₅₁H₇₉NO₁₃ and its molecular weight is 914.172 g/mol. Its structure is shown below. Isomers of rapamycin are known, e.g., isomer B and isomer C, having structures as shown in U.S. Pat. No. 7,384,953. Typically, rapamycin is a mixture of the B and C isomers. In solution, rapamycin isomers B and C interconvert and an equilibrium is achieved. It is common practice in the literature to depict the structure of rapamycin in the form of the B isomer, which is the form shown below.

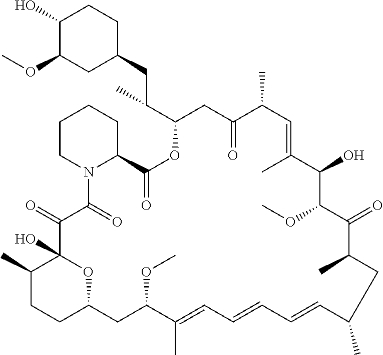 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Rapamycin is a white to off-white powder and is considered insoluble in water, having a very low solubility of only 2.6 μg/mL. It is freely soluble in benzyl alcohol, chloroform, acetone, and acetonitrile. The water insolubility of rapamycin presents special technical problems to its formulation. In the context of its formulation as an oral dosage form, it has been prepared as an oral solution in the form of a solid dispersion (WO 97/03654) and a tablet containing nanosized (less than 400 nm) particles (U.S. Pat. No. 5,989,591). But these procedures suffer from substantial variation in the dissolution of the active and therefore its bioavailability. Another method of formulation utilizes the crystalline powder. According to art-recognized methods, the transformation of the crystalline form of a low solubility drug to its amorphous form can significantly increase its solubility. While this is also true for rapamycin, the amorphous form is extremely chemically unstable. Pharmaceutical dosage forms including amorphous rapamycin (sirolimus) are described in WO 06/039237 and WO 06/094507 (modified release formulation including sirolimus and glyceryl monostearate at a concentration of 49.25%). An improved stable oral dosage form of rapamycin is described in U.S. Pat. No. 8,053,444. The dosage form employs a fatty acid ester and a polymer (e.g., polyvinylpyrrolidone (PVP), hydroxypropylcellulose (HPC) or hydroxypropylmethyl cellulose (HPMC)) in the composition to increase the stability of sirolimus without adversely affecting its release rate. According to U.S. Pat. No. 8,053,444, a fatty acid ester concentration exceeding 10% w/w suppresses the release rate of sirolimus from the formulation and so should be avoided because it can lead to insufficient absorption from the gastrointestinal tract. The preferred concentration of fatty acid ester (glycerol ester) is 1% to 5% or 5% to 9%. In one aspect, the aerosol combination formulations of the present invention do not contain a fatty acid ester in combination with a polymer. In one aspect, the aerosol combination formulations of the disclosure contain a fatty acid ester at a concentration exceeding 10% or exceeding 12% by weight of the composition.

Rapamycin and its derivatives (including analogs) and prodrugs suitable for use in the compositions, products and methods of the disclosure include rapamycin (sirolimus) and prodrugs or derivatives thereof which are inhibitors of the mTOR cellular signaling pathway, and preferably inhibitors of mTOR itself. In one aspect a rapamycin derivative or prodrug is an mTOR inhibitor selected from the group consisting of everolimus (Affinitor; RAD001), temsirolimus (CCI-779), ridaforolimus (previously known as deforolimus; AP23573), umirolimus (Biolimus A9), zotarolimus (ABT-578), novolimus, myolimus, AP23841, KU-0063794, INK-128, EX2044, EX3855, EX7518, AZD08055 and OSI027. Further derivatives are known to the skilled person and include, for example, an O-substituted derivative in which the hydroxyl group on the cyclohexyl ring of sirolimus is replaced by —OR1, in which R₁ is optionally a substituted alkyl, acylaminoalkyl or aminoalkyl.

In one embodiment, the present disclosure provides a pharmaceutical composition or product including rapamycin or an analog thereof, treprostinil or an analog thereof, and a carrier. In one aspect, the rapamycin compound for use in the aerosol formulations and methods of the disclosure is a rapamycin derivative (analog) selected from the group consisting of analogs everolimus, temsirolimus, ridaforolimus, umirolimus, and zotarolimus. The chemical structures of the rapamycin analogs everolimus, temsirolimus, ridaforolimus, umirolimus, and zotarolimus are shown below.

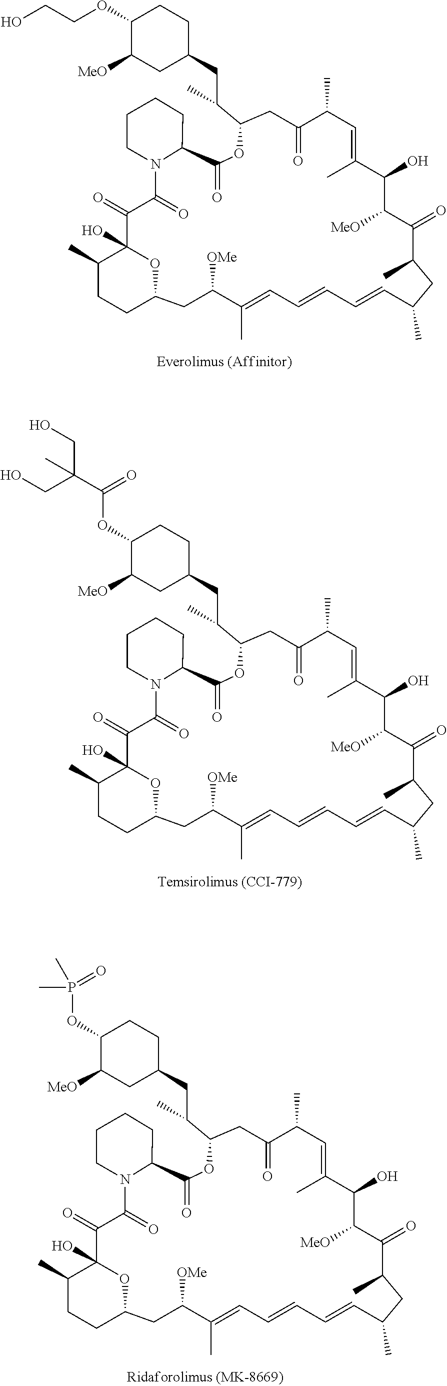

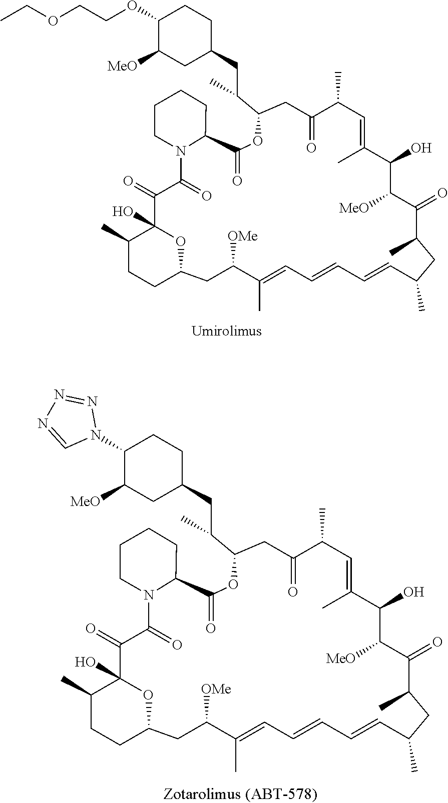 [SMILES: CCOCCO[C@@H]1CC[C@@H](C[C@@H](C)[C@@H]2CC(=O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)C[C@H]1OC ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](n3cnnn3)[C@H](OC)C2)CC(=O)[C@H](C)/C=C(\C)[C@@H](O)[C@@H](OC)C(=O)[C@H](C)C[C@H](C)/C=C/C=C/C=C/1C]

In one aspect, the compound for use in the aerosol formulations, products and methods of the disclosure is an mTOR inhibitor selected from the group consisting of KU-0063794, AZD8055, INK128, and OSI-027. The chemical structures of the mTOR inhibitors KU-0063794, AZD8055, INK128, and OSI-027 are shown below.

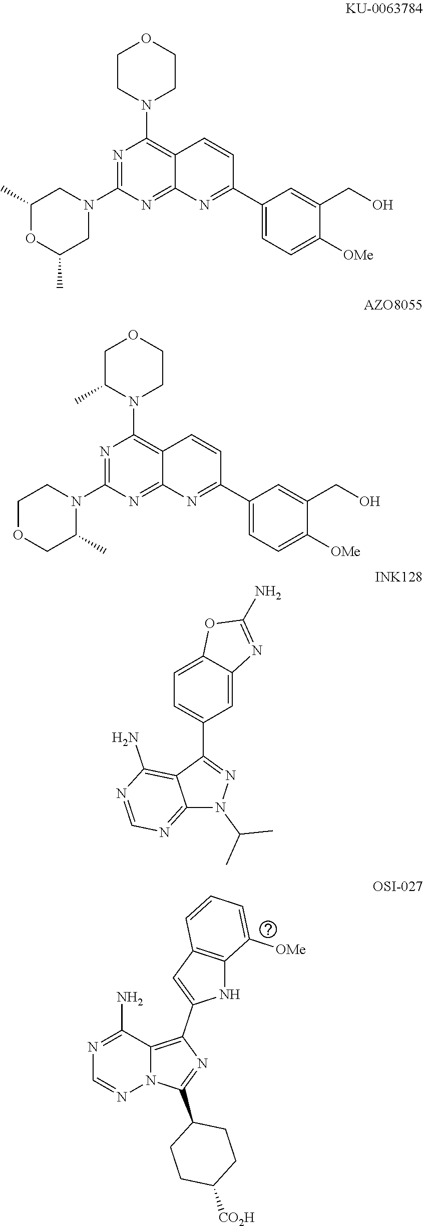

Particularly preferred for use in the methods, products and compositions of the disclosure are sirolimus, temsirolimus, and everolimus. In one aspect, the compound is sirolimus or everolimus.

### Treprostinil

Treprostinil is a synthetic prostacyclin analog, with the formula shown below. Its molecular formula is C23H3405 and has a molecular weight of 390.5 g/mol. The chemical (IUPAC) name of treprostinil is ({(1R,2R,3aS,9aS)-2-Hydroxy-1-[(3S)-3-hydroxyoctyl]-2,3,3a,4,9,9a-hexahydro-1H-cyclopenta[b]naphthalen-5-yl}oxy) acetic acid.

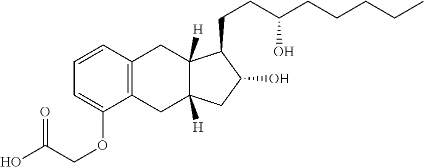 [SMILES: CCCCC[C@H](O)CC[C@@H]1[C@H]2Cc3cccc(OCC(=O)O)c3C[C@H]2C[C@H]1O]

Treprostinil is considered to have low solubility in water, with a solubility of only 6 μg/mL, while treprostinil salts, such as treprostinil sodium, can be soluble in water. Treprostinil sodium is soluble in water and ethanol, slightly soluble in acetone, and has low solubility in acetonitrile, n-hexane, and ethyl acetate. An improved stable oral dosage form of treprostinil employs a fumaryl diketopiperazine (FDKP) in the composition to facilitate formation of a powder formulation. Treprostinil and its derivatives (including analogs) and prodrugs suitable for use in the compositions, products and methods of the disclosure include treprostinil and prodrugs or derivatives thereof, which are inhibitors of the prostacyclin pathway, and preferably inhibitors of prostacyclin itself. In one aspect, a treprostinil derivative or prodrug is selected from the group including various prodrugs and derivatives designed to improve its pharmacokinetic properties, such as absorption and excretion.

Treprostinil is one of the treatments for pulmonary arterial hypertension (PAH) as it dilates the pulmonary arteries and reduces the pressure in the lungs. The inhibition of mTORC1 (mTOR-Raptor) by treprostinil suggests that it not only serves as a vasodilator but also possesses antiproliferative properties. Treprostinil can be delivered by inhalation, which avoids the need for continuous infusion or subcutaneous injection. It can also be delivered orally. Different inhalation formulations of treprostinil have different characteristics, such as duration of effect and pharmacokinetics. YUTREPIA® (Liquidia) is a dry powder inhalation formulation of treprostinil, developed using PRINT® technology for efficient deep-lung delivery. TYVASO® (United Therapeutics) is another inhalation formulation of treprostinil, available as a solution or a dry powder inhaler (TYVASO® DPI). INS1009 (TPIP; Insmed) is a dry powder inhalation formulation of treprostinil palmitil, a prodrug of treprostinil, designed for sustained release.

YUTREPIA® requires dosing four times daily, similar to TYVASO®, due to its short half-life. TYVASO® also necessitates four daily doses because of its short half-life of about four hours. In contrast, INS1009 (TPIP) is designed for once-daily dosing, providing a longer duration of effect compared to YUTREPIA® and TYVASO®. YUTREPIA® exhibits rapid absorption with peak plasma concentration typically reached within 10-15 minutes post-inhalation. TYVASO® has a similar rapid absorption profile, with peak plasma concentration also within 10-15 minutes. INS1009 (TPIP) shows a lower maximum plasma concentration and sustained levels of treprostinil over 12 hours, allowing for once-daily dosing.

TABLE 2
Comparison of Different Formulations of Treprostinil

| | Tmax | Cmax | AUC | T½ | |
|---|---|---|---|---|---|
| Drug | (hours) | (ng/mL) | (ng · h/mL) | (hours) | Notes |
| TYVASO ® | ~0.25 | ~0.5-1.0 | ~2.5-3.0 | ~4-5 | Rapid absorption and elimination; |
| DPI | | | | | designed for frequent dosing. |
| YUTREPIA ® | ~0.5 | ~1.0-1.5 | ~3.0-4.0 | ~4-6 | Optimized for pulmonary delivery |
| | | | | | with consistent systemic exposure. |
| INS1009 | ~1.0 | ~0.8-1.2 | ~4.0-5.0 | ~8-10 | Extended half-life due to prodrug |
| | | | | | formulation, supporting once-daily |
| | | | | | dosing. |
| Oral | ~4 | ~2.5 | ~25 | ~4-6 | Slower absorption; requires |
| Treprostinil | | | | | multiple daily doses for sustained |
| | | | | | effect. |
| Nebulized | ~0.25 | ~0.5-1.0 | ~2.5-3.0 | ~4-5 | Rapid absorption; localized |
| treprostinil | | | | | delivery to lungs minimizes |
| | | | | | systemic exposure. |
| DPI | ~0.25-0.5 | ~1.0-1.5 | ~3.0-4.0 | ~4-6 | Optimized for pulmonary delivery |
| Treprostinil | | | | | with consistent systemic exposure. |

link.springer.com/article/10.1007/s40262-016-0409-0; www.accessdata.fda.gov/drugsatfda_docs/nda/2022/214324Orig1s000lbl.pdf.

All three drugs have a generally well-tolerated safety profile, with side effects that are common and consistent with the known effects of prostacyclin. YUTREPIA® has common side effects including cough, headache, and throat irritation, consistent with known prostacyclin side effects. TYVASO® shares a similar safety profile, with common side effects such as cough, throat irritation, headache, and dizziness. INS1009 (TPIP) has been well-tolerated in clinical trials, with most side effects being mild and consistent with those reported for other treprostinil-based therapies.

TABLE 3
Side Effects of Treprostinil

| | | | Major Safety | |
|---|---|---|---|---|
| Formulation | Study Name | Indication | Signals | Notes |
| Oral | FREEDOM-EV | Pulmonary | Headache, | Common |
| Treprostinil | | Arterial | nausea, diarrhea, | prostacyclin- |
| | | Hypertension | jaw pain, and | related side |
| | | (PAH) | flushing | effects; generally |
| | | | | manageable with |
| | | | | dose adjustments. |
| Nebulized | INCREASE | Pulmonary | Cough, throat | Localized |
| Treprostinil | | Hypertension | irritation, | delivery to lungs |
| | | associated with | headache, | minimized |
| | | Interstitial | dizziness, and | systemic side |
| | | Lung Disease | nausea | effects. |
| | | (PH-ILD) | | |
| DPI | BREEZE | Pulmonary | Cough, throat | Transition from |
| Treprostinil | | Arterial | irritation, | nebulized |
| | | Hypertension | headache, and | TYVASO ® to |
| | | (PAH) | dizziness | DPI was |
| | | | | generally well- |
| | | | | tolerated. |
| Inhaled | TETON-PPF | Progressive | Cough, throat | Safety profile |
| Treprostinil | | Pulmonary | irritation, and | consistent with |
| | | Fibrosis (PPF) | mild respiratory | other inhaled |
| | | | symptoms | formulations. |

ir.unither.com/~/media/Files/U/United-Therapeutics-IR/documents/press-releases/2023/2023-10-31-ppf-fpfv-final.pdf.

Treprostinil is generally well-tolerated, but liver-related concerns have been noted in specific cases. It is contraindicated in patients with severe liver disease, as impaired liver function can affect the drug's metabolism and clearance. Patients with mild to moderate liver impairment may require dose adjustments to avoid potential toxicity. In rare cases, treprostinil has been associated with elevated liver enzymes, which could indicate liver stress or damage.

Treprostinil is an inhaled prostacyclin mimetic used to treat pulmonary hypertension (PH). The mechanism of action includes several pathways. Treprostinil promotes the direct dilation of pulmonary and systemic arterial vascular beds, helping to reduce high blood pressure in the lungs associated with PH. Additionally, it inhibits platelet aggregation, which can help prevent blood clots that might worsen PH. Treprostinil also inhibits the proliferation of smooth muscle cells in the pulmonary arteries, which can help prevent the narrowing of these vessels. By targeting these key pathways, treprostinil helps improve blood flow and reduce the symptoms of pulmonary hypertension.

Treprostinil is a potent agonist for the DPI receptor but is not highly selective. It also has significant activity at other prostanoid receptors, including the IP (prostacyclin) and EP2 receptors. Treprostinil has a high binding affinity for the DP1 receptor, with a reported Ki value of approximately 4.4 nM, indicating strong interaction with the DP1 receptor. In addition to DP1, treprostinil shows high affinity for the IP and EP2 receptors, with Ki values of 32 nM and 3.6 nM, respectively. In functional assays, treprostinil has been shown to elevate cyclic AMP levels with high potency in cells expressing DP1, IP, and EP2 receptors, indicating its strong agonistic activity at these sites. These points highlight that while treprostinil is a potent DP1 agonist, it is not highly selective and also activates other prostanoid receptors, contributing to its overall pharmacological profile (see Table 4 and Table 5 below (compiled from Whittle B. J. et al. “Binding and Activity of the Prostacyclin Receptor (IP) Agonists, Treprostinil and Iloprost, at Human Prostanoid Receptors: Treprostinil is a Potent DP1 and EP2 Agonist” Mar. 27, 2012 Biochemical Pharmacology 83:68-75; “DP1 Receptor, Prostanoid Receptors” IUPHAR/BPS Guide to Pharmacology, retrieved from the internet <URL: www.guidetopharmacology.org/GRAC/ObjectDisplayForward?objectId=338; and Zeng et al. “Prostaglandin and Prostaglandin Receptors: present and future promising therapeutic targets for pulmonary arterial hypertension; Respiratory Research, vol. 24, 263 (2023) See also, Chapman_2020_An overview of the biology of a long-acting inhaled Treprostinil Prodrug; Fiorentu_2025_Pulmonary Hypertension Associated with Interstitial Lung Disease: Back to the Future; and Lambers_2018_Treprostinil inhibits proliferation and extracellular matrix deposition by fibroblasts through cAMP activation.

TABLE 4
Comparison of Binding Affinity between Different
Prostacyclins and Prostacyclin Receptor Agonists
for Various Prostaglandin Receptors.

| | Receptors | | | | | | | |
|---|---|---|---|---|---|---|---|---|
| Drugs | IP | EP1 | EP2 | EP3 | EP4 | DP1 | FP | TP |

Prostacyclin and prostacyclin analogs

| | | | | | | | | |
|---|---|---|---|---|---|---|---|---|
| Epoprostenol | ++++ | ++ | / | ++++ | N | / | / | ++ |
| Iloprost | ++++ | ++++ | + | +++ | ++ | + | ++ | + |
| Treprostinil | ++++ | ++ | ++++ | + | ++ | ++++ | + | N |
| Beraprost | +++ | N | N | ++ | + | N | N | N |

Prostacyclin receptor agonist

| | | | | | | | | |
|---|---|---|---|---|---|---|---|---|
| Selexipag | ++++ | N | N | N | N | N | N | N |

“+”: affinity strength. The greater the number, the stronger the affinity.
“N”: no affinity.
“/” data not available.

TABLE 5
A Summarization of Prostaglandin Receptors Signaling in PH

| | Functional | | |
|---|---|---|---|
| Targets | Cell Types | Mechanism | Phenotypic Features |
| DP1 | VSMCs | cAMP/PKA | Vasodilation |
| | PASMCs | PKA/raptor-dependent | Inhibit proliferation |
| | | mTCRC1 dissociation | and hypertrophy |
| DP2 | Th2 | Release IL-4, IL-13 | Promote PASMCs |
| | | | proliferation by |
| | | | activating STAT6 |
| EP1 | VSMCs | PLC/PKC | Vasoconstriction |
| EP2 | VSMCs | cAMP/PKA | Vasodilation |
| EP3 | VSMCs | Inhibit cAMP/PKA | Vasoconstriction |
| | PASMCs | Rho-dependent | Extracellular matrix |
| | | extracellular | remodeling |
| | | MMP-2/TGF-β1 | |
| EP4 | VSMCs | cAMP/PKA | Vasodilation |
| | Pasmcs | PKA/PPARγ | Inhibit PASMCs |
| | | | proliferation |
| | | | and migration |
| | PASMCs | Kv channels | Vasodilation |
| IP | VSMCs | cAMP/PKA | Vasodilation |
| TP | VSMCs | PLC/PKC | Vasoconstriction |
| | PASMCs | Rho | Vasoconstriction |
| | PASMCs | Inhibit Kv channels | Inhibit proliferation |

In vitro models have shown that treprostinil exerts several beneficial effects on pulmonary artery endothelial cells (PAECs), which manage pulmonary arterial hypertension (PAH). Treprostinil induces vasodilation by stimulating the production of cyclic adenosine monophosphate (cAMP) in PAECs, leading to the relaxation of the pulmonary arteries, and reducing pulmonary vascular resistance. It also has anti-proliferative effects, inhibiting proliferation of PAECs and smooth muscle cells by modulating the mTOR signaling pathway, helping to prevent the vascular remodeling that characterizes PAH. Additionally, treprostinil reduces inflammation in the pulmonary arteries by decreasing the expression of pro-inflammatory cytokines and adhesion molecules in PAECs. It supports improved endothelial function by increasing the release of nitric oxide (NO) and prostacyclin, both of which are vasodilators and inhibitors of platelet aggregation.

Treprostinil acts on pulmonary artery smooth muscle cells (PASMCs) through several mechanisms that help manage pulmonary arterial hypertension (PAH). One of the primary actions of treprostinil is inducing vasodilation by stimulating the production of cyclic adenosine monophosphate (CAMP) in PASMCs. This leads to the relaxation of the pulmonary arteries and a reduction in pulmonary vascular resistance. Additionally, treprostinil inhibits the proliferation of PASMCs by modulating the mTOR signaling pathway, which helps prevent the vascular remodeling that characterizes PAH.

Furthermore, treprostinil reduces inflammation in the pulmonary arteries by decreasing the expression of pro-inflammatory cytokines and adhesion molecules in PASMCs. This anti-inflammatory action helps to manage the inflammatory component of PAH. Lastly, treprostinil enhances endothelial function by increasing the release of nitric oxide (NO) and prostacyclin, both of which are vasodilators and inhibitors of platelet aggregation. These combined effects contribute to the overall therapeutic benefits of treprostinil in treating PAH.

In preclinical studies for TYVASO® (treprostinil) targeting idiopathic pulmonary fibrosis (IPF), bleomycin-induced pulmonary fibrosis models in mice were used (journals.sagepub.com/doi/pdf/10.1177/2045894019881954). Bleomycin, a chemotherapeutic agent, is administered to mice through various routes, such as intratracheal, intraperitoneal, or intravenous injections to induce lung fibrosis. This method helps researchers evaluate the antifibrotic and anti-inflammatory effects of potential treatments like TYVASO®.

In vitro studies of human hepatic microsomes (www.tyvaso.com/pdf/TYVASO-PI.pdf) showed that treprostinil does not inhibit cytochrome P450 (CYP) isoenzymes CYP1A2, CYP2A6, CYP2C8, CYP2C9, CYP2C19, CYP2D6, CYP2E1, and CYP3A. Human pharmacokinetic studies with an oral formulation of treprostinil (treprostinil diolamine) indicated that co-administration of the cytochrome P450 (CYP) 2C8 enzyme inhibitor, gemfibrozil, increases exposure (both Cmax and AUC) to treprostinil. Co-administration of the CYP2C8 enzyme inducer, rifampin, decreases exposure to treprostinil. It is unclear if the safety and efficacy of treprostinil by the inhalation route are altered by inhibitors or inducers of CYP2C8.

TYVASO® (treprostinil) has undergone extensive preclinical studies to support its development, particularly for treating conditions like idiopathic pulmonary fibrosis (IPF) and progressive pulmonary fibrosis (PPF). These studies have demonstrated that treprostinil possesses significant antifibrotic properties, which help reduce the formation of fibrous tissue, a hallmark of diseases such as idiopathic pulmonary fibrosis (IPF) and progressive pulmonary fibrosis (PPF). Data from the INCREASE study showed that nebulized TYVASO® led to significant improvements in forced vital capacity (FVC), a key measure of lung function, particularly notable in patients with underlying IPF. Furthermore, open-label extension data indicated that these improvements in FVC were sustained for at least 64 weeks, suggesting long-term benefits of the treatment. These preclinical findings provided a strong foundation for the clinical trials and eventual approval of TYVASO® for treating pulmonary hypertension associated with interstitial lung disease (PH-ILD). Ongoing studies are also exploring its efficacy in broader fibrotic lung diseases. See for example, ir.unither.com/press-releases/2025/02-04-2025-120131472; ir.unither.com/~/media/Files/U/United-Therapeutics-IR/documents/press-releases/2023/2023-10-31-ppf-fpfv-final.pdf.

YUTREPIA® has demonstrated significant improvements in exercise capacity and quality of life in patients with pulmonary arterial hypertension (PAH) and pulmonary hypertension associated with interstitial lung disease (PH-ILD). Similarly, TYVASO® has proven efficacy in enhancing exercise capacity and quality of life in patients suffering from PAH and PH-ILD. INS1009 (TPIP) has demonstrated favorable outcomes in clinical trials, showing significant improvements in both pulmonary vascular resistance and exercise capacity. See for example, link.springer.com/article/10.1007/s12325-022-02296-x The following information regarding TYVASO is directly from the FDA approved label. TYVASO® DPI (dry powder inhaler) contains the active ingredient treprostinil in the form of an inhalation powder. It is available in single-dose cartridges containing 16 mcg, 32 mcg, 48 mcg, or 64 mcg of treprostinil.

The initial dose for TYVASO® DPI is one 16 mcg cartridge per treatment session, administered four times daily. The dose can be increased by an additional 16 mcg per treatment session at approximately 1- to 2-week intervals, if tolerated. The target maintenance doses range from 48 ug to 64 ug per treatment session, administered four times daily.

TYVASO® DPI should be used only with the TYVASO® DPI Inhaler. Each cartridge is administered using a single inhalation. Four separate treatment sessions should be conducted each day, approximately four hours apart during waking hours and the dose can be a fixed dose. As used herein, a fixed dose combination product or formulation refers to a combination of two or more active drugs in a single dosage form.

L606 (LIQUIDIA®) represents the next generation of treprostinil formulations, where the drug is encapsulated within liposomes.

A combination of mTOR inhibition and prostacyclin therapy offers potential synergistic improvement in treatment through various effective mechanisms. In PAH, DP1 expression is downregulated in PASMCs, and loss of DP1 leads to upregulation of mTORC1 activity. Reduction of DP1 limits the actions of treprostinil and further limits the ability to act on the mTOR pathway. Rapamycin circumvents the signaling limitation caused by treprostinil. Formulation considerations include that the combination of treprostinil with rapamycin may result in a longer pharmacodynamic response in endothelial cells (ECs) or smooth muscle cells (SMS).

In some aspects, the treprostinil compound for use in the formulations, products and methods presented herein is a treprostinil derivative, prodrug or analog selected from the group consisting of treprostinil diolamine, treprostinil sodium, treprostinil phosphate, or treprostinil palmitil. In some aspects, the treprostinil compound is a compound described in U.S. Pat. Nos. 12,173,021, 10,716,793, or 12,173,071, the contents of which are incorporated herein in their entirety. In some aspects, the treprostinil compound is selected from the group consisting of:

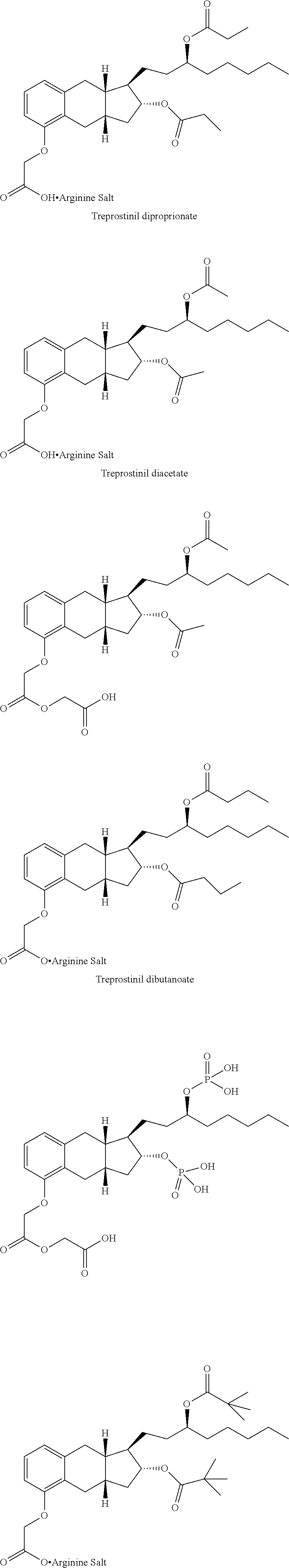

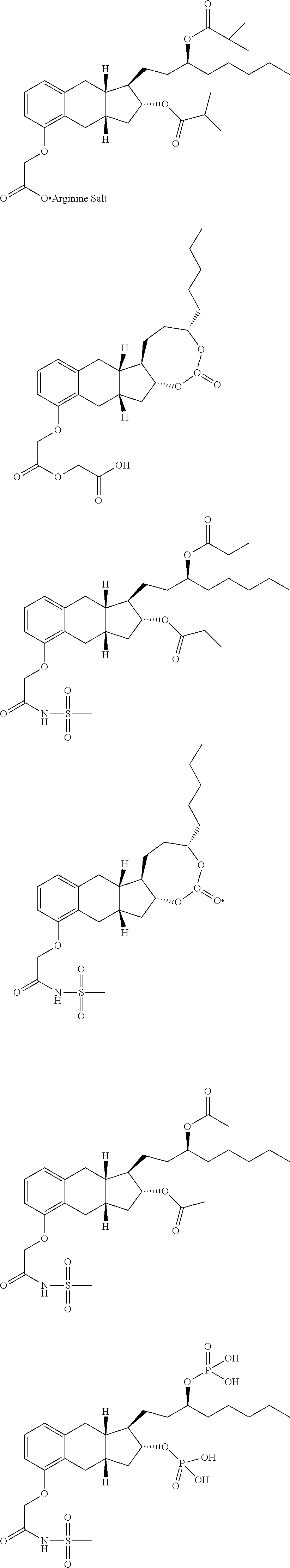

Treprostinil and rapamycin may provide complementary therapeutic effects when administered in combination, either as a single formulation, a fixed dose combination product or formulation or as separate formulations. The vasodilatory and anti-proliferative properties of treprostinil work synergistically with the immunosuppressive and anti-inflammatory effects of rapamycin to address multiple aspects of diseases such as pulmonary arterial hypertension and other cardiopulmonary disorders. In some cases, co-administration of these agents allows for lower doses and or lower frequency of each individual drug while maintaining or enhancing overall efficacy. The combination also mitigates certain side effects associated with higher doses of either drug alone. As an illustrative example, rapamycin and Treprostinil may be dosed QD or BID, and optionally, Treprostinil would be dosed yet again in a single day (e.g., one, two or more times). For example, currently Yutrepia requires dosing four times daily, similar to Tyvaso, due to its short half-life (e.g., 4 hours), while INS1009 is designed for once-daily dosing, providing a longer duration of effect relative to Yutrepia and Tyvaso. One of skill in the art and the physician will be able to determine the appropriate dosing and frequency schedule for a patient based on the teachings herein and knowledge of those of skill in the art based on these approved drugs as examples.

Various modes of administration, including oral, subcutaneous, and inhalation routes, for example, are employed to deliver the combination therapy or monotherapies, with the choice of formulation and delivery method potentially impacting the pharmacokinetics and therapeutic outcomes.

In some aspects, the method inhibits biological activity of mTORC1 in the lungs. In some aspects, the method inhibits phosphorylation of the S6 protein in the lungs. In some aspects, the method inhibits platelet aggregation, dilates pulmonary and systemic arterial vascular beds, or inhibits smooth muscle cell proliferation. In some aspects, the method interferes with the cell cycle progression of vascular smooth muscle cells (VSMCs) by elevating expression of inhibitor of DNA binding 1 (ID1).

In some aspects, the lung disease or disorder treated with the methods and composition provided herein is pulmonary hypertension, idiopathic pulmonary fibrosis, progressive pulmonary fibrosis, right heart failure secondary to pulmonary hypertension, lymphangioleiomyomatosis, pulmonary capillary hemangiomatosis, or hereditary hemorrhagic telangiectasia. In some aspects, the pulmonary hypertension is pulmonary arterial hypertension, pulmonary hypertension associated with interstitial lung disease, chronic thromboembolic pulmonary hypertension, or sarcoidosis-associated pulmonary hypertension.

### Compositions for Inhalation

The present disclosure provides pharmaceutical compositions and products adapted for administration by inhalation, including rapamycin or an analog thereof and treprostinil or an analog thereof in the form of an aqueous solution, a dry powder, or a mixture of one or more pharmaceutically acceptable propellants and a carrier. In some aspects, the composition or product is formulated for administration by inhalation or oral delivery. In one aspect, the rapamycin and treprostinil are encapsulated in a pharmaceutically acceptable compound, material, or matrix. In one aspect, the rapamycin and treprostinil are encapsulated in a liposomal formulation or a non-liposomal formulation.

The compositions and products of the disclosure include aerosolizable formulations of rapamycin and treprostinil suitable for pulmonary drug delivery in a subject by inhalation of the aerosol. The term “aerosol” is used in this context to mean a colloidal system in which the dispersed phase is composed of either solid or liquid particles and in which the dispersal medium is a gas. In one aspect, the gas is air, and the formulation is a solution formulation suitable for administration via a nebulizer or a dry powder formulation suitable for administration via a dry powder inhaler device. Generally, respirable particles or droplets will have a mean diameter in the range of 0.10 to 10 microns. The size of the particles or droplets is selected to maximize targeted delivery either to the lungs themselves (i.e., where the lung is the target tissue) or systemically (where the lungs are utilized as an alternative route for systemic administration). The size will preferably be in the range of about 0.5 to 5 microns where the lung itself is the therapeutic target, or less than 3 microns for systemic delivery via the lung. Size is measured according to methods known in the art and described, for example, in the U.S. Pharmacopeia at Chapters 905 and 601. For example, it is measured as Mass Median Aerodynamic Diameter (MMAD). In one aspect, the average or mean diameter of the particles, including the compositions and products described herein, is measured as MMAD.

In one aspect, the dispersed phase of the aerosol is composed of liquid particles or droplets. In this context, the terms “liquid particles” and “droplets” are used interchangeably. In this aspect, the formulation of the present disclosure is a solution formulation. In one aspect, the dispersed phase of the aerosol is composed of solid particles. In this aspect, the formulation of the invention is a dry powder formulation. Micronized particles of this size can be produced by methods known in the art, for example by mechanical grinding (milling), precipitation from subcritical or supercritical solutions, spray-drying, freeze-drying, or lyophilization.

Generally, inhaled particles are subject to deposition by one of two mechanisms: impaction, which usually predominates for larger particles, and sedimentation, which is prevalent for smaller particles. Impaction occurs when the momentum of an inhaled particle is large enough that the particle does not follow the air stream and encounters a physiological surface. In contrast, sedimentation occurs primarily in the deep lung when very small particles which have traveled with the inhaled air stream encounter physiological surfaces as a result of random diffusion within the air stream. The aerosol formulations of the invention are preferably adapted to maximize their deposition either by impaction (in the upper airways) or by sedimentation (in the alveoli), in order to achieve the desired therapeutic efficacy.

The amount of drug delivered to the patient from a delivery device, such as a nebulizer, pMDI or DPI device, is referred to as the delivered dose. It can be estimated in vitro by determining the amount of drug emitted from the delivery device in a simulated inhalation maneuver. This is termed emitted dose (ED) as measured according to methods known in the art, for example those set out in the U.S. and European Pharmacopeias, e.g., at Chapter 601 and Chapter 905 of the USP. Accordingly, “emitted dose” is considered equivalent to the delivered dose.

The amount of drug delivered from the delivery device to the lungs of the patient is termed the respirable dose. It can be estimated in vitro by determining the fine particle dose (FPD) as measured using cascade impactors, such as a Next Generation Impactor (NGI) according to methods known in the art, for example those set out in the U.S. and European Pharmacopeias, e.g., at Chapters 601 and 905 of the USP.

The amount of drug released in fine, inhalable particles from a delivery device is referred to as the fine particle fraction (FPF) of the formulation. FPF is the fraction of drug in the delivered dose that is potentially respirable. Thus, FPF is the ratio of fine particle dose (FPD) to emitted or delivered dose (ED). These characteristics of the formulation are measured according to methods known in the art, for example those set out in the U.S. and European Pharmacopeias, e.g., at Chapter 601 of the USP and monograph 2.9.18 of the Pharm Europa.

In some aspects, the composition achieves a respirable dose of rapamycin from 1 to 500 micrograms and a respirable dose of treprostinil from 1 to 500 micrograms delivered to the lung. In some aspects, the respirable dose of rapamycin is about 5, about 20, about 50, about 100, or about 250 micrograms. In some aspects, the respirable dose of treprostinil is about 16, about 48, about 64, about 84, about 150, or about 250 micrograms.

In one aspect, the aerosolizable rapamycin in the combination formulation has an FPF greater than 20% with a corresponding FPD ranging from 10 micrograms to 2 milligrams, preferably less than 0.5 milligrams, even after prolonged storage, e.g., after 1 to 12 months or after 1 to 36 months of storage. In one aspect, the dose delivered to the patient, the delivered dose (DD) or emitted dose (ED), ranges from 25 micrograms to 2.5 milligrams, preferably less than 0.5 milligrams.

In some aspects, the aerosolizable treprostinil in the combination formulation has an FPF greater than 20% with a corresponding FPD ranging from 10 micrograms to 2 milligrams, preferably less than 0.5 milligrams, even after prolonged storage, e.g., after 1 to 12 months or after 1 to 36 months of storage. In one aspect, the dose delivered to the patient, the delivered dose (DD) or emitted dose (ED) of treprostinil ranges from 25 micrograms to 2.5 milligrams, preferably less than 0.5 milligrams.

In certain aspects, the rapamycin and treprostinil are encapsulated in a pharmaceutically acceptable compound, material, or matrix. In one aspect, the rapamycin and treprostinil are encapsulated in a liposomal formulation or non-liposomal formulation.

### Aqueous Solution Compositions

In one aspect, the aerosolizable composition or product of the disclosure is an aqueous solution formulation of rapamycin and treprostinil adapted for pulmonary delivery via a nebulizer, including jet, vibrating mesh, and static mesh or orifice nebulizers. Thus, the solution formulation is adapted to enable aerosol droplet formation in the respirable range of from about 0.1 to 10 microns diameter, as described above. In one aspect, the composition is a nebulizable aqueous solution formulation consisting of rapamycin (sirolimus) or an analog thereof dissolved in water, treprostinil or an analog thereof, ethanol, and a low molecular weight polyol, and optionally including a surface active agent. In one aspect, the aqueous solution formulation has a viscosity below 20 mPa-s, below 10 mPa-s, or below 5 mPa-s, and a surface tension of at least 45 dynes/cm, preferably greater than 60 dynes/cm. Preferably, the formulation has a viscosity below 5 mPa-s, and a surface tension above 45 dynes/cm. In one aspect, the composition has a viscosity below 20 mPa-s, a viscosity below 10 mPa-s, or a viscosity below 5 mPa-s and a surface tension of at least 45 dynes/cm, preferably greater than 60 dynes/cm.

In one aspect, the aqueous solution formulation consists of rapamycin, treprostinil, water, ethanol, and a low molecular weight polyol selected from glycerol and propylene glycol. In one aspect, the aqueous solution formulation consists of rapamycin, treprostinil, water, and a low molecular weight polyol selected from glycerol and propylene glycol, with the ethanol being optional. The formulation may also optionally contain a non-ionic surfactant, preferably PEG 100, or a polysorbate, preferably Polysorbate 80 (“PS80”), a phospholipid, preferably a natural phospholipid such as lecithin, and preferably hydrogenated soya lecithin, and an antioxidant or stabilizer, preferably disodium EDTA. In one aspect, the non-ionic surfactant is selected from the group consisting of polyethylene glycol (PEG) PEG 100, PEG 1000, and Polysorbate 80 (also referred to as Tween™ 80, sorbitan monooleate, or polyoxyethylene sorbitan oleate), and mixtures thereof.

In some aspects, the administration includes microparticles of rapamycin and microparticles of treprostinil, particles of a carrier, and optionally at least one excipient, wherein the composition is effective to deliver a therapeutic amount of the composition to the lungs. In some aspects, an amount of the rapamycin in the composition is from about 0.1% to 20% (w/w). In some aspects, the amount of the rapamycin in the composition is from about 0.25% to 2% (w/w), based upon the total weight of the composition. In some aspects, an amount of the treprostinil in the composition is from about 0.01% to 20% w/w. In some aspects, the amount of the treprostinil in the composition is from about 0.25% to 2% based upon the total weight of the composition.

In one aspect, the concentration of rapamycin in solution is from about 0.01 to 0.1 mg/ml, the concentration of treprostinil is from about 100 to 3000 ug/mL, the amount of the low molecular weight polyol is from 5 to 35% w/w, the amount of ethanol is present in the amount of 5-20% w/w, and the amount of the non-ionic surfactant is from 1 to 200 parts per million (ppm) w/w. Preferably, the amount of non-ionic surfactant is less than 100 ppm (w/w). The amounts of the optional anti-oxidant/stabilizer are from zero to less than 0.01% w/w.

In one aspect, the aqueous solution formulation of the disclosure does not contain one or more additives or excipients selected from the group consisting of polyethylene glycol, lecithin, EDTA, a block copolymer, and a cyclodextrin.

The aqueous solution formulation is a single phase aqueous solution in which the rapamycin is completely dissolved. The main co-solvents in the formulation are ethanol and a low molecular weight polyol selected from glycerol and propylene glycol. The rapamycin is not in suspension or emulsion, nor can the solution be described as a colloidal solution or dispersion. The aqueous solution formulation of the present disclosure lacks colloidal structures such as micelles or liposomes. The amount of phospholipid, if present, is too small to form liposomes or to precipitate the rapamycin. And the combined amount of phospholipid and non-ionic surfactant is too small to modify surface tension.

Neither the phospholipid nor the non-ionic surfactant is present in amounts sufficient to act as a surfactant in the traditional sense. In this context, the term surfactant refers to an agent that acts to lower the surface tension of the solution or the interfacial tension between the liquid and any solid drug particles in solution such that the surfactant acts as a detergent, wetting agent, emulsifier, or dispersing agent. Instead, the non-ionic surfactant in the solution formulation of the invention serves to block adsorption of the drug to the polyethylene container in which the final product is packaged, thereby preventing loss of drug potency via adsorption to the container.

Accordingly, in one aspect, the aqueous solution formulation is a single-phase aqueous solution in which the rapamycin is completely dissolved, the solution lacks micelles or liposomes, and the solution is not an emulsion, dispersion, or suspension.

In one aspect, the solution formulation is sterile. In one aspect, the solution formulation is sterile-filtered through a 0.2 micron filter. In one aspect, the solution formulation is not sterilized by heat, such as by autoclaving, or by radiation.

In one aspect, the disclosure provides a package containing one or more containers or vials (these terms are used interchangeably) filled with the sterile aqueous solution formulation. Preferably, the containers are unit dose containers. In one aspect, the containers are polymer vials, preferably polyethylene vials. In one aspect, the container or vial filled with the sterile aqueous solution formulation of the disclosure is produced by a process including the steps of forming the vial by blow molding and immediately thereafter filling the vial with the sterile-filtered formulation of the invention under aseptic conditions, followed by thermal sealing of the vial immediately after it is filled.

In one aspect, the aqueous aerosol formulation of the disclosure includes the following:
rapamycin (or an analog thereof) from about 0.001% to 0.01% w/w,
treprostinil (or an analog thereof) from about 0.001% to 0.1% w/w,
propylene glycol from about 5% to 35% w/w,
ethanol from about 5% to 20% w/w,
polysorbate 80 from about 1 to 200 ppm w/w,
lecithin from about 1 to 100 ppm w/w,
and water,
   where the amount of water is sufficient to achieve a concentration of the rapamycin between 0.01 and 0.1 milligrams/milliliter and a concentration of treprostinil between 0.01 μg/mL and 2 ng/mL. Optionally, a stability enhancer could be added, such as disodium EDTA at levels below 0.01% w/w.

For aqueous and other non-pressurized liquid systems, a variety of nebulizers (including small-volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Healthcare, Inc. and DeVilbiss Health Care, Inc.

In one aspect, the aqueous aerosol formulation of the disclosure is delivered via a vibrating nebulizer available from Aerogen, Pari, Philips, or Omron. In one aspect, the aqueous aerosol formulation of the invention is packaged in a container suitable for use with a vibrating mesh nebulizer, for example, the AERONEB® Go (Aerogen, distributed by Philips Respironics), I-NEB® (Philips) or E-FLOW® (Pari), or similar nebulizer. In one aspect the aqueous aerosol formulation of the disclosure is delivered via an orifice nebulizer such as the RESPIMAT® from Boehringer-Ingelheim.

Thus, in one aspect the disclosure provides a pharmaceutical composition or product in the form of a nebulizable aqueous solution suitable for administration by inhalation to a subject, the aqueous solution consisting of rapamycin or an analog thereof, preferably selected from sirolimus, everolimus, and temsirolimus; treprostinil or an analog thereof; water, ethanol, and a low molecular weight polyol. In one aspect, the low molecular weight polyol is glycerol or propylene glycol, or a mixture thereof. In one aspect, the composition further includes a nonionic surfactant selected from the group consisting of PEG 100, PEG 1000, polysorbate 80, and mixtures thereof. In one aspect, the amount of nonionic surfactant in the formulation is from 1 to 200 ppm w/w, preferably less than 100 ppm w/w, based upon the weight of the formulation. In one aspect, the composition further includes a phospholipid, an antioxidant or chemical stabilizer. In one aspect, the amount of antioxidant or chemical stabilizer in the formulation is less than 0.01% w/w based upon the weight of the formulation. In one aspect, the antioxidant or chemical stabilizer is EDTA. In one aspect, the amount of rapamycin in the formulation is from 0.001 to 0.01% w/w based upon the weight of the formulation. In one aspect, the amount of treprostinil in the formulation is from 0.001 to 0.1% w/w based upon the weight of the formulation.

In one aspect, the composition does not contain one or more additives or excipients selected from the group consisting of polyethylene glycol, lecithin, EDTA, a block copolymer, and a cyclodextrin.

In one aspect, the composition lacks colloidal structures selected from micelles and liposomes.

In one aspect, the composition is suitable for administration via any one of a jet nebulizer, a vibrating mesh nebulizer, a static mesh nebulizer, and an orifice nebulizer.

In one aspect, the composition has a viscosity below 20 mPa-s, preferably below 10 mPa-s, most preferably below 5 mPa-s, and a surface tension of at least 45 dynes/cm, preferably at least 50 dynes/cm.

The disclosure also provides a method of manufacturing a pharmaceutical composition of the invention in the form of a nebulizable aqueous solution, the method including sterile filtering the solution through a filter with pore size of 0.2 microns or less and collecting the sterile filtrate in collection vessel under aseptic conditions. In one aspect, the method of manufacturing further includes transferring the sterile filtrate into a container closure under aseptic conditions. In one aspect, the container closure is a unit-dose polyethylene vial. In one aspect, the vial is produced by blow-molding immediately before the sterile filtrate is transferred to the vial. In one aspect, the method further includes the step of thermally sealing the vial immediately after the sterile filtrate is transferred to the vial.

### Dry Powder Compositions

In one aspect, the aerosolizable composition or product of the disclosure is a dry powder including micronized particles of rapamycin or a prodrug or derivative thereof and micronized particles of treprostinil or a prodrug or derivative thereof. In some aspects, the particles have diameters from 0.1 to 10 microns and a mean diameter of between about 0.5 to 4.5 microns, about 1 to 4 microns, about 1 to 3.5 microns, about 1.5 to 3.5 microns, or about 2 to 3 microns. The dry powder formulation is suitable for use in either a dry powder inhaler device (DPI) or a pressurized metered dose inhaler (pMDI). The amount of rapamycin in the dry powder is from about 0.5 to 20% (w/w) based on total weight of the powder. In one aspect, the amount of rapamycin is about 1% or 2% (w/w). The amount of treprostinil in the dry powder is from about 0.1 to 50% (w/w) based on the total weight of the powder. In one aspect, the amount of treprostinil is about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% w/w based on the total weight of the powder.

In one aspect, micronized rapamycin or treprostinil is produced by wet polishing or jet milling as described below to generate diameters in the range of about 0.5 to 4.5 microns, about 1 to 4 microns, or about 2 to 3 microns, and the particles are blended onto lactose carrier particles in a drug/carrier ratio ranging from 0.52% w/w with a preferred ratio of 1%. The particles may include rapamycin, treprostinil, or a combination thereof. In some aspects, the ratio of the rapamycin to the carrier in the powder is 1% w/w. In some aspects, the ratio of the treprostinil to the carrier in the powder is 1% w/w. In one aspect, micronized rapamycin is produced by wet polishing or jet milling, micronized treprostinil is produced by wet polishing or jet milling, and the micronized rapamycin and micronized treprostinil are combined in the final formulation. In another aspect, the rapamycin and treprostinil are combined, then the rapamycin and treprostinil combination is micronized, and the micronized rapamycin and treprostinil are added to the final formulation. The micronized active agents form drug particles that are added to the combination formulation.

In some aspects, a first carrier includes particles having diameters ranging from about 30-100 microns, and a second carrier includes particles having diameters of less than 10 microns. In some aspects, the ratio of the two different carriers is in the range of from 95-98:2-5.

In one aspect, the drug particles are lightly compacted into a frangible matrix which is contained within the delivery device (a dry powder inhaler). Upon actuation, the delivery device abrades a portion of the drug particles from the matrix and disperses them in the inspiratory breath, delivering the drug particles to the respiratory tract. Alternatively, the drug particles may be a free-flowing powder contained within a reservoir in the delivery device (a dry powder inhaler). The reservoir can be an integral chamber within the device, or a capsule, blister or similar preformed reservoir that is inserted into the device prior to actuation. Upon actuation, the device disperses a portion of the drug particles from the reservoir and disperses them in the inhalation breath delivering the drug particles to the respiratory tract.

In some aspects, the composition includes a carrier. In some aspects, the carrier is selected from the group consisting of arabinose, glucose, fructose, ribose, mannose, sucrose, trehalose, lactose, maltose, starches, dextran, mannitol, lysine, leucine, isoleucine, dipalmitoylphosphatidylcholine, lecithin, polylactic acid, poly(lactic-co-glutamic) acid, and xylitol, or mixtures thereof.

In one aspect, the dry powder composition consists of drug particles and a carrier selected from the group consisting of arabinose, glucose, fructose, ribose, mannose, sucrose, trehalose, lactose, maltose, starches, dextran, mannitol, leucine, lysine, isoleucine, dipalmitoylphosphatidylcholine, lecithin, polylactic acid, poly(lactic-co-glutamic) acid, and xylitol, or mixtures of any of the foregoing. In one aspect, the carrier is lactose, particularly in the form of the monohydrate. In some aspects, the carrier includes a blend of two different carriers. In some aspects, the carrier includes a blend of two different lactose carriers.

The term “lactose carriers” refers to a carrier commonly used in dry powder inhalation (DPI) formulations or compositions. Examples of lactose carriers include but are not limited to anhydrous lactose, lactose monohydrate, spray-dried lactose, crystalline lactose, and granulated lactose.

In one aspect the dry powder composition includes drug and a blend of at least two different carriers. In one aspect, the drug to carrier ratio is in the range of from about 0.5 to 20% (w/w). In one aspect, the drug particles have diameters ranging from 0.1 to 10 microns with a mean diameter of about 1 to 4, 1 to 3.5, or 1.5 to 3.5, or 2 to 3 microns. The carrier particles may have diameters ranging from 2 to 200 microns.

In one aspect, the composition is contained in a blister pack or a reservoir of a DPI device. In one aspect, the dry powder composition is preloaded into a gelatin, starch, cellulosic, or polymeric capsule, or a foil/foil or foil/plastic blister suitable for use in a DPI device. Each capsule or blister may contain from 1 to 100 milligrams of the dry powder composition. The capsules or blisters may be inserted into a dry powder inhaler (DPI) device such as AEROLIZER®, PLASTIAPE® RS01 Model 7, and PLASTIAPE® RSOO Model 8, XCAPS®, FLOWCAPS®, ARCUS®, DISKHALER® or MICRODOSE®. Upon actuating the DPI device, the capsules or blisters are ruptured, and the powder is dispersed in the inspiratory breath, delivering the drug to the respiratory tract.

In one aspect, the dry powder composition is contained in a dry powder inhaler (DPI) device selected from ACCUHALER®, CONIX™, ROTAHALER®, TWINCAPS®, XCAPS®, FLOWCAPS®, TURBUHALER®, NEXTHALER®, CYCLOHALER®, REVOLIZER™, DISKHALER®, DISKUS®, SPINHALER, HANDIHALER®, Microdose Inhaler, GYROHALER®, OMNIHALER®, CLICKHALER®, DUOHALER® (Vectura), and ARCUS® inhaler (Civitas Therapeutics). In one aspect, the invention provides a DPI device containing a dry powder composition described herein. In one aspect the device is selected from the group consisting of XCAPS®, FLOWCAPS®, HANDIHALER®, TWINCAPS®, AEROLIZER®, PLASTIAPE® RS01 Model 7, and PLASTIAPE® RSOO Model 8. In one aspect, the device containing the composition is selected from the group consisting of a GYROHALER®, an OMNIHALER®, a CLICKHALER®, a DUOHALER®, and an ARCUS® inhaler.

The carrier particles are preferably of larger size (greater than 5 microns) so as to avoid deposition of the carrier material in the deep lung. In one aspect, the carrier particles have diameters ranging from 1 to 200 microns, from 30 to 100 microns, or less than 10 microns. In one aspect the carrier particles are a blend of two carriers, one with particles of about 30-100 microns and the other with particles less than 10 microns. The ratio of the two different carriers is in the range of from 3:97 to 97:3. In one aspect, the dry powder composition consists of 0.5 to 20% (w/w) drug to carrier ratio, the drug particles having diameters from 0.1 to 10 microns with a mean diameter less than 3.5 microns. In one aspect, the carrier material is a crystalline carrier material. Preferably, the crystalline carrier material is one which is at least 90%, preferably greater than 95% crystalline and in which no or substantially no water is absorbed by the carrier under conditions of 80% or lower relative humidity at room temperature. Examples of such crystalline carriers are lactose monohydrate and glucose monohydrate. The amount of carrier is from 1 to 99.0% or more of the formulation by dry weight of the powder, preferably 5 to 99%, 10 to 99%, 20 to 99%, 30 to 99%, 40 to 99%, or 50 to 99%.

In one aspect, the dry powder composition is contained within a reservoir in the delivery device (a dry powder inhaler). The reservoir can be an integral chamber within the device, or a capsule, blister or similar preformed reservoir that is inserted into the device prior to actuation. Upon actuation, the device disperses a portion of the drug particles from the reservoir and disperses them in the inspiratory breath delivering the drug particles to the respiratory tract.

In one aspect, the drug is present as a fine powder with a pharmaceutically acceptable carrier. In this context, the term “fine” refers to a particle size in the inhalable range, as discussed above. Preferably, the drug is micronized such that the particles have a mean diameter in the range of 10 microns or less. In some aspects, the microparticles of rapamycin have a mass aerodynamic diameter of about 0.1 to 10 microns and a mean mass aerodynamic diameter (MMAD) of about 1.5 to 4 microns. In some aspects, the microparticles of rapamycin have an MMAD of about 1.5 to 3.5 microns, or about 2 to 3 microns. In some aspects, the microparticles of treprostinil have a range of mass aerodynamic diameter between about 0.1 to 10 microns and an MMAD of about 1 to 4 microns. In some aspects, the microparticles of treprostinil have a range of MMAD between about 1.5 to 3.5 microns, or about 2 to 3 microns.

In one aspect, the dry powder formulation of rapamycin and treprostinil further includes one or more additives selected from the additives described below. In some aspects, at least one excipient is present and is selected from a phospholipid and a salt of a fatty acid. In some aspects, the phospholipid is selected from dipalmitoylphosphatidylcholine and lecithin. In some aspects, the salt of a fatty acid is magnesium stearate. In one aspect of this aspect, the magnesium stearate is present in amounts of 0.001 to 10% by dry weight of the powder, preferably in amounts of from 0.01 to 5% or 0.01 to 2%. In another aspect, the additive includes or consists of a phospholipid, such as lecithin (which is a mixture of phosphatidylcholines) in an amount of 0.1% to 1% by dry weight of the powder, preferably 0.2% to 0.6%. In one aspect of this aspect, the additive is coated onto the carrier material prior to or simultaneously with a step of blending the carrier with the particles of rapamycin and the particles of treprostinil. In some aspects, the at least one excipient is coated on the carrier particles in a weight ratio of excipient to large carrier particle ranging from 0.01 to 0.5%. This can be accomplished, for example, by utilizing a high energy mixing step to coat the carrier with the additive, or a long duration of low energy mixing, or a combination of low and high energy mixing to achieve the desired level of coated carrier material. Low energy devices for mixing dry powders to form blends are known in the art and include, for example, V-blenders, double cone blenders, slant cone blenders, cube blenders, bin blenders, horizontal or vertical drum blenders, static continuous blenders, and dynamic continuous blenders. Other, higher energy devices include high-shear mixers known to those skilled in the art.

In certain aspects, the dry powder is contained in a capsule. In one aspect the capsule is a gelatin capsule, a plastic capsule, or a cellulosic capsule, or is in the form of a foil/foil or foil/plastic blisters. In each instance, the capsule or blister is suitable for use in a DPI device, preferably in dosage units together with the carrier in amounts to bring the total weight of powder in each capsule to from 1 mg to 100 mg. Alternatively, the dry powder may be contained in a reservoir of a multi-dose DPI device.

The particle size of the drug can be reduced to the desired microparticulate level by conventional methods, for example by grinding in an air-jet mill, ball mill, or vibrator mill, by wet polishing, microprecipitation, spray drying, lyophilization or recrystallization from subcritical or supercritical solutions. Jet milling or grinding in this context refers to micronization of dry drug particles by mechanical means. Micronization techniques do not require making a solution, slurry, or suspension of the drug. Instead, the drug particles are mechanically reduced in size. Due to the relatively high energy that is employed by micronization, in certain aspects it is desirable to include a carrier material in a co-micronized mixture with the rapamycin, treprostinil, or combination thereof. In this context, the carrier material absorbs some of the energy of micronization which otherwise could adversely affect the structure of the rapamycin, treprostinil, or combination thereof. In one aspect, rapamycin particle, treprostinil particles, or particles of a combination of rapamycin and treprostinil in a size range of from 1 to 4 or from 2 to 3 microns are produced by a jet milling method.

Wet polishing, as described in US2013/0203717, involves using high shear to reduce the particle size of the drug particles in a suspension or slurry. Wet polishing can include just the drug particles or additional particulates termed milling media. In one aspect, the particle size of the rapamycin, treprostinil, or combination thereof can be reduced to the desired level using a wet polishing process, which includes wet milling, specifically by cavitation at elevated pressure, where rapamycin, treprostinil, or combination thereof is suspended in water or other solvent where it is insoluble, and then is followed by spray drying of the suspension to obtain rapamycin, treprostinil, or combination thereof as a dry powder. In one aspect, particles of rapamycin, treprostinil, or combinations thereof in a size range of from 1 to 4 or from 2 to 3 microns are produced by a wet polishing method that includes preparing a suspension of rapamycin, subjecting the suspension to microfluidization, and spray-drying the resulting particles to form a dry powder. The rapamycin, treprostinil, or combination thereof may be suspended in an anti-solvent selected from the group consisting of propyl or butyl alcohol, water, and ethyl acetate. In one aspect, the suspension is an aqueous suspension.

The term “microfluidization” refers to a high-shear fluid processing technique used to create fine emulsions, suspensions, and nanoparticles. Microfluidization involves passing fluids at high pressure through micro-channels, creating intense shear, impact, and cavitation forces. This results in the reduction of particle size and the formation of stable emulsions or suspensions.

Spray drying generally involves making a solution, slurry, or suspension of the drug, atomizing the solution, slurry, or suspension, to form particles and then evaporating the solution, slurry, or suspension media to form the particles. The solution, slurry, or suspension can be formed under subcritical or supercritical conditions. The evaporation step can be accomplished by elevating the temperature of the atmosphere into which the atomization occurs, or by decreasing the pressure, or a combination of both. In one aspect, the powder formulation including rapamycin, treprostinil, or a combination thereof is made by spray drying an aqueous dispersion of rapamycin to form a dry powder consisting of aggregated particles of rapamycin, treprostinil, or a combination thereof having a size suitable for pulmonary delivery, as described above. The aggregate particle size can be adjusted (increased or decreased) to target either the deep lung or upper respiratory sites, such as the upper bronchial region or nasal mucosa. This can be accomplished, for example, by increasing the concentration of rapamycin, treprostinil, or a combination thereof in the spray-dried dispersion or by increasing the droplet size generated by the spray dryer.

Alternatively, the dry powder can be made by freeze-drying (lyophilization) the aqueous drug solution, dispersion, or emulsion or by a combination of spray-drying and freeze-drying.

In one aspect, the aqueous dispersion of rapamycin, treprostinil, or a combination thereof and the one or more optional additives further includes a dissolved diluent such as lactose or mannitol such that when the dispersion is freeze-dried, respirable diluent particles, each containing at least one embedded drug particle and additive particle, if present, are formed.

In one aspect, the dry powder formulation is made by freeze-drying an aqueous dispersion of rapamycin, treprostinil, or a combination thereof, and one or more optional additives. In one aspect, the powders contain aggregates of rapamycin, treprostinil, or combination thereof and an additive, if present, wherein the aggregates are within a respirable size range as described above.

In one aspect, the dry powder includes liposomes loaded with rapamycin, treprostinil, or a combination thereof. Drug-loaded liposomes can be produced by methods known in the art, for example using the technique described for tacrolimus in M. Chougale, et al. Int. J. Nanomedicine 2:625-688 (2007). Briefly, rapamycin, treprostinil, or a combination thereof, hydrogenated phosphatidylcholine (HSPC), and cholesterol are dissolved in a mixture of methanol and chloroform and then subjected to dry thin film formation, e.g., in Rotaevaporator. The liposomes are hydrated and the liposomal dispersion is passed through a high-pressure homogenizer for size reduction. The resultant pellets are characterized for vesicle size and percent drug entrapment and pellets equivalent to the desired amount of rapamycin, treprostinil, or combination thereof are then dispersed in a suitable medium and subjected to spray-drying to obtain particles of the desired size for inhalation. The spray-dried powder can be filled into capsules, canisters, or blister packs for administration.

In one aspect, the dry powder particles can be produced by precipitation from a supercritical or subcritical solution.

The dry powder compositions may be contained in a suitable dry powder inhaler device, or in a capsule or blister for use in such a device. Examples of such devices are provided above and include ACCUHALER®, AEROLIZER®, THE PLASTIAPE® RS01 Model 7, the PLASTIAPE® RS00 Model 8, CONIX™, ROTAHALER®. TWINCAPS®, XCAPS®, FLOWCAPS®, TURBUHALER®, NEXTHALER®, CYCLOHALER®, REVOLIZER™, DISKHALER®, DISKUS®, SPINHALER, HANDIHALER®, Microdose Inhaler, GYROHALER®, OMNIHALER®, CLICKHALER®, OR DUOHALER® (Vectura), or a breath-actuated ARCUS® inhaler (Civitas Therapeutics). In one aspect, the invention provides a DPI device containing a dry powder composition described herein. In one aspect the device is selected from the group consisting of XCAPS®, FLOWCAPS®, HanHANDIHALER®, dihaler, TWINCAPS®, AEROLIZER®, the PLASTIAPER RS01 Model 7, and the PLASTIAPE® RSOO Model 8.

### Propellant-Based Formulations

In another aspect of the disclosure, the rapamycin and treprostinil are formulated in a propellant-based formulation, which may also be referred to generically herein as “a pMDI formulation.” A pMDI formulation is suitable for delivery by a device such as a pressurized metered dose inhaler (pMDI). In one aspect, the composition includes rapamycin and treprostinil, a propellant, and a vegetable oil or pharmaceutically acceptable derivative of a vegetable oil. The propellant is preferably selected from 1,1,1,2-tetrafluoroethane (HFA134a) and 1,1,1,2,3,3,3-heptafluoropropane (HFA227), or mixtures thereof. In one aspect, the vegetable oil is selected from olive oil, safflower oil, and soybean oil. The rapamycin, treprostinil, or combination thereof may be in solution or in suspension in the propellant. In this context, “in suspension” refers to where the rapamycin, treprostinil, or combination thereof is present in particulate form dispersed in the propellant. In one aspect, the rapamycin, treprostinil, or combination thereof is micronized and is present in suspension in the propellent. In one aspect, the formulation further includes a wetting agent or co-solvent such as ethanol. In one aspect, the formulation further includes a polyhydroxy alcohol such as propylene glycol.

Suitable propellants are known in the art and include, for example, halogen-substituted hydrocarbons, for example fluorine-substituted methanes, ethanes, propanes, butanes, cyclopropanes or cyclobutanes, particularly 1,1,1,2-tetrafluoroethane (HFA134a) and 1,1,1,2,3,3,3-heptafluoropropane (HFA227), or mixtures thereof.

In one aspect, the formulation includes micronized rapamycin, micronized treprostinil, ethanol, a suitable propellant such as HFA 134a, HFA 227, or a mixture of suitable propellants, and optionally one or more surfactants. In one aspect, the formulation further includes a lubricant.

In one aspect, the formulation includes rapamycin, micronized treprostinil, a propellant, and a vegetable oil. In one aspect, the formulation does not include an additive or surfactant. For example, the formulation does not include ethanol, a polyhydroxy alcohol (e.g., propylene glycol), or a surfactant (e.g., sorbitan trioleate, sorbitan monooleate, or oleic acid).

In one aspect, the propellant-based formulation includes compressed air, carbon dioxide, nitrogen or a liquefied propellant selected from the group consisting of n-propane, n-butane, isobutane or mixtures thereof, or 1,1,1,2-tetrafluoroethane (HFA134a) and 1,1,1,2,3,3,3-heptafluoropropane (HFA227), or mixtures thereof, with or without a polar co-solvent such as an alcohol. The composition can be a solution or a suspension. For suspensions, the drug particles have diameters from 0.1 to 10 microns with a mean diameter of less than 3.5 microns.

The propellant-based formulation is prepared by methods known in the art, for example by wet milling the coarse rapamycin and optional additives in liquid propellant, either at ambient pressure or under high-pressure conditions. The treprostinil is also wet milled with an optional additive in liquid propellant either at ambient pressure or under high pressure. In some aspects, the rapamycin and treprostinil are mixed prior to wet milling. In certain aspects, the additive is a surfactant which serves to prevent aggregation (caking or crystallization), to facilitate uniform dosing, and (or alternatively) to provide a favorable fine particle fraction (FPF). In one aspect, the surfactant is selected from sorbitan trioleate, sorbitan monooleate, or oleic acid. Alternatively, dry powders containing drug particles are prepared by spray-drying or freeze-drying aqueous dispersions of the drug particles as discussed above, and the resultant powders are dispersed into suitable propellants for use in conventional pressurized metered dose inhalers (pMDIs). In one aspect, the inhalation device is a RESPIMAT™

In one aspect, the propellant-based aerosol rapamycin and treprostinil formulations of the invention are stable against particle size growth or change in the crystal morphology of the rapamycin, treprostinil, or a combination thereof over prolonged periods of time.

### Process for Manufacturing Sterile Unit Dose Forms

In one aspect, the compositions of the disclosure are sterile compositions. In one aspect, the sterile compositions are sterile unit dose forms. In one aspect, the sterile unit dosage form is a capsule suitable for use in a nebulizer device.

In one aspect, the finished composition is sterilized in its container-closure by heat, e.g., autoclaving, or by radiation. In one aspect, the component parts of the composition are first sterilized by a suitable process including sterile filtration for liquid components and radiation or autoclaving for solids or liquids, the process further including maintaining the sterility of the sterile components by packaging in hermetic containers, combining the components in a mixing vessel in the appropriate proportions, and filling the resulting product into a container closure, all performed in an aseptic suite. This process has the disadvantage of being expensive and requiring difficult aseptic handling techniques. Accordingly, it is used primarily to process particulate suspensions or colloidal dispersions, liposomal formulations, or emulsions, which cannot be passed through a submicron filter for sterilization. Finally, in one aspect, the finished composition is sterile filtered through a submicron filter, preferably a 0.2 micron filter. In one aspect, the compositions, and products of the disclosure are single-phase aqueous solutions sterilized via a filtration sterilization process. In contrast, emulsions and liposomal formulations are typically not sufficiently stable under the high shear conditions of a filtration sterilization process and so are not preferred for this process. In one aspect, the compositions, and products of the disclosure are single-phase aqueous solutions which are filled into a container-closure, e.g., a vial, formed of a polymer, preferably polyethylene, or alternatively a glass vial. Autoclaving and radiation are not suitable where the vial is a polymer vial because of the high likelihood of creating chemical instability in the drug and/or formulation excipients, as well as in the container, and due to the generation of undesirable impurities. In one aspect, the compositions and products of the disclosure are sterilized by a process that does not include heat (autoclaving) or radiation, and instead includes a filtration sterilization process. Preferably, in accordance with this aspect, the single-phase aqueous solutions of rapamycin and treprostinil are sterilized by filtration through a filter having a pore size less than or equal to 0.2 microns. In one aspect, the sterile filtrate is collected in a collection vessel located in an aseptic suite. In one aspect, the sterile filtrate is transferred from the collection vessel into a container closure in an aseptic suite. Preferably the container closure is a polymer vial, preferably a unit dose vial, and most preferably a polyethylene unit dose vial. In one aspect, the polymer vial is formed by blow-molding immediately before it is filled and then thermally sealed immediately after filling. This technique may be also referred to as “form-fill-seal” or a “blow-fill.” This technique is particularly advantageous in the context of the compositions of the invention, which are single-phase aqueous solutions of rapamycin, treprostinil, or a combination thereof because this process does not require heat or radiation, both of which may degrade either the drug itself, the formulation excipients, or the container closure.

### Pulmonary Administration and Dosing

The present disclosure provides compositions and methods for the treatment and prophylaxis of lung diseases and disorders in a subject by administering rapamycin and treprostinil to the respiratory tract, preferably to the lungs, by inhalation. Pulmonary delivery is preferably accomplished by inhalation of the aerosol through the mouth and throat into the lungs but may also be accomplished by inhalation of the aerosol through the nose. Thus, in one aspect, the composition or product is delivered intranasally. In another aspect, the aerosol is delivered perorally.

The compositions, products and methods of the disclosure advantageously provide for the targeted delivery of a therapeutically effective amount of rapamycin and treprostinil to the lungs while simultaneously reducing to very low or undetectable levels the amount of rapamycin and treprostinil in the blood and available systemically. In one aspect, the amount of rapamycin in a single dose of a dry powder composition described herein is from about 5 to 500 micrograms or from about 100 to 300 micrograms, or from about 50 to 250 micrograms. The targeted delivery of low dose rapamycin directly to the lungs while minimizing systemic exposure provides for an improved therapeutic index compared to oral dosage forms.

In one aspect, the amount of treprostinil in a single dose of a dry powder composition described herein is from about 5 to 3000 micrograms, or from about 50 to 1000 micrograms, or from about 100 to 1000 micrograms. The targeted delivery of low dose rapamycin directly to the lungs while minimizing systemic exposure provides for an improved therapeutic index compared to oral dosage forms.

In one aspect, administration of rapamycin and treprostinil by inhalation according to the methods of the disclosure increases the therapeutic index of rapamycin, treprostinil, or a combination thereof. In this context, the therapeutic index is a ratio that compares the dose that produces a therapeutic effect (ED₅₀) to the dose that produces a toxicity (TD₅₀) in 50% of the population. The ratio is represented as TD₅₀/ED₅₀. In one aspect, administration of rapamycin and treprostinil by inhalation according to the methods of the invention reduces one or more toxicities associated with orally administered rapamycin, treprostinil, or a combination thereof, thereby increasing the therapeutic index of rapamycin, treprostinil, or a combination thereof.

The disclosure includes aerosolizable formulations in the form of solutions and powders. Accordingly, the rapamycin, treprostinil, or a combination thereof may be administered according to the methods of the invention in the form of an aqueous aerosol, a dry powder aerosol, or a propellant-based aerosol.

In some aspects, the rapamycin is present at an amount effective to produce a concentration in the lung tissue of from 1 ng/g to 1 ug/g. In some aspects, the rapamycin is present at an amount that produces a blood trough level of less than 5 ng/mL, less than 2 ng/mL, less than 1 ng/mL, less than 0.5 ng/ml, or less than 0.25 ng/mL. In some aspects, the treprostinil is present in an amount effective to produce a concentration in the lung tissue of from 1 ng/g to 1 ug/g. In some aspects, the treprostinil is present in an amount that produces a blood trough level of less than 5 ng/ml, less than 2 ng/ml, less than 1 ng/mL, less than 0.5 ng/ml, or less than 0.25 ng/mL.

In one aspect, the administered dose of rapamycin is from 5 to 100 micrograms, from 20 to 100 micrograms, from 20 to 250 micrograms, from 50 to 500 micrograms (0.05 to 0.5 milligrams), from 250 to 1000 micrograms (0.25 to 1 milligrams) or from 500 to 2000 micrograms (0.5 to 2 milligrams). In one aspect, the amount of rapamycin administered is less than 500 micrograms, less than 100 micrograms, less than 50 micrograms, less than 20 micrograms, or less than 10 micrograms. Preferably, the amount of rapamycin administered is less than 0.5 milligrams or less than 0.25 milligrams.

In some aspects, the composition is formulated for once daily, twice daily, three times daily, or four times daily or more administration. In some aspects, the composition is formulated for once daily administration. In some aspects, a total daily dose of rapamycin administered is from 20 ug to 20 mg. In some aspects, a total daily dose of treprostinil administered is from 1 ug to 20 mg.

In one aspect, the total daily dose of rapamycin is in the range of from 5 to 100 micrograms, from 20 to 250 micrograms, from 50 to 500 micrograms (0.05 to 0.5 milligrams), from 250 to 1000 micrograms (0.5 to 1 milligrams) or from 500 to 2000 micrograms (0.5 to 2 milligrams). In one aspect, the total daily dose of rapamycin is less than 500 micrograms, less than 100 micrograms, less than 50 micrograms, less than 20 micrograms, or less than 10 micrograms. In one aspect, the total daily dose of rapamycin administered to the subject is less than 0.5 milligrams or less than 0.25 milligrams per day.

In one aspect, a composition of the disclosure is administered once per day to the subject. In one aspect, a composition of the disclosure is administered twice or three times a day. Preferably, the composition or product is administered once or twice daily, or less than once daily.

In one aspect, the methods of the disclosure include administering rapamycin via a pulmonary route in combination with one or more additional therapeutic agents. The one or more additional agents may be administered by the same or a different route of administration as the rapamycin. For example, the agent may be administered by inhalation, intranasally, orally, or intravenously.

In one aspect, the methods of the disclosure include administering rapamycin via a pulmonary route in combination with one or more additional therapies.

In certain aspects, the methods include pulmonary administration of a composition of the invention as the primary therapy. In other aspects, the administration of a composition of the disclosure is an adjuvant therapy. In either case, the methods of the invention contemplate the administration of a composition or product of the disclosure in combination with one or more additional therapies for the treatment of a disease or disorder. The terms “therapy” and “therapies” refer to any method, protocol and/or agent that can be used in the prevention, treatment, management or amelioration of a disease or disorder, or one or more symptoms thereof.

Preferably, the administration of a pharmaceutical composition or product including rapamycin or a prodrug or derivative thereof according to the methods of the disclosure in combination with one or more additional therapies provides a synergistic response in the subject. In this context, the term “synergistic” refers to the efficacy of the combination being more effective than the additive effects of either single therapy alone. In one aspect, the synergistic effect of combination rapamycin therapy according to the invention permits the use of lower dosages and/or less frequent administration of at least one therapy in the combination compared to its dose and/or frequency outside of the combination. In some aspects, a dose of treprostinil administered in combination with rapamycin is lower than a dose of treprostinil administered in the absence of rapamycin. In another aspect, the synergistic effect is manifested in the avoidance or reduction of adverse or unwanted side effects associated with the use of either therapy in the combination alone.

### Nebulizer Delivery

In one aspect, the rapamycin or treprostinil is formulated as an aqueous solution suitable for nebulization and delivered via a nebulizer. For aqueous and other non-pressurized liquid systems, a variety of nebulizers (including small volume nebulizers) are available to aerosolize the formulations. Compressor-driven nebulizers incorporate jet technology and use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., De Vilbiss Health Care, Inc.; and Hospitak, Inc. Ultrasonic nebulizers rely on mechanical energy in the form of vibration of a piezoelectric crystal to generate respirable liquid droplets and are commercially available from, for example, Omron Healthcare, Inc. and DeVilbiss Health Care, Inc. The nebulizer may be, for example, a conventional pneumatic nebulizer such as an airjet nebulizer, or an ultrasonic nebulizer, which may contain, for example, from 1 to 50 mL, commonly 1 to 10 mL, of the solution formulation.

In one aspect, the aqueous solution formulation of the disclosure is adapted for administration with a nebulizer including a vibrating or fixed mesh. For example, devices such as an AERX® (Aradigm), RESPIMAT® (Boehringer Ingelheim), I-NEB® (Philips), or MICROAIRE® (Omron) in which drug solution is pushed with a piston or pneumatic pressure, or with a piezoelectric crystal through an orifice or mesh. Alternatively, the solution can be pumped through a vibrating mesh nebulizer such as the E-FLOW® (Pari) or AERONEB® Go (Aerogen). These devices allow much smaller nebulized volumes, e.g., 10 to 100 μl, and higher delivery efficiencies than conventional nebulizers.

### Dry Powder Delivery

In one aspect, the dry powder compositions of the disclosure are delivered by a non-propellant based dry powder inhaler (DPI) device. In one aspect, the powder is contained in capsules of gelatin or plastic, or in blisters, suitable for use in a DPI device. In one aspect, the powder is supplied in unit dosage form and in dosage units of from 5 mg to 100 mg of powder per capsule. In another aspect, the dry powder is contained in a reservoir of a multi-dose dry powder inhalation device. In one aspect, the inhaler device includes an aerosol vial provided with a valve adapted to deliver a metered dose, such as 10 to 100 μl, e.g., 25 to 50 μl, of the composition, i.e., a device known as a metered dose inhaler.

In one aspect, the DPI device is a blister-based device such as the GYROHALER® or the OMNIHALER® (both from Vectura), a reservoir-based device such as the CLICKHALER® or DUOHALER® (Vectura), and the ARCUS® inhaler (Civitas Therapeutics). In one aspect, the DPI device is selected from PULMATRIX™ and Hovione TWINCAPS® and XCAPS™. In one aspect the device is selected from the group consisting of XCAPS™, PLASTIAPE® RS01 Model 7, and PLASTIAPER RSOO Model 8.

In one aspect, the DPI device is selected from the group consisting of ACCUHALER®, AEROLIZER®, the PLASTIAPER RS01 Model 7, the PLASTIAPER RSOO Model 8, CONIX™, ROTAHALER®, TWINCAPS®, XCAPS®, FLOWCAPS®, TURBUHALER®, NEXTHALER®, CYCLOHALER®, REVOLIZER™, DISKHALER®, DISKUS®, SPINHALER®, HANDIHALER®, Microdose Inhaler, GYROHALER®, OMNIHALER®, CLICKHALER®, or DUOHALER® (Vectura), or a breath-actuated ARCUS® inhaler (Civitas Therapeutics).

In one aspect, the DPI device is selected from the group consisting of ARCUS™, ASPIRAIR™, AXAHALER™, BREEZHALER™, CLICKHALER™ CONIX DRY™, CRICKET™, DREAMBOAT™, GENUAIR™, GEMINI™ INSPIROMATIC™, ISPERSE™, MICRODOSE™, NEXT DPI™, PROHALER™ PULMOJET™, PULVINAL™, SOLIS™, TAIFUN™, TAPER DRY™, TRIVAI™ NOVOLIZER™, PODHALER™, SKYEHALER™, SPIROMAX™, TWINCAPS/FLOWCAPS™, and TURBUHALER™. In one aspect, the DPI device is adapted to deliver the dry powder from a capsule or blister containing a dosage unit of the dry powder or a multi-dose dry powder inhalation device adapted to deliver, for example, 5-25 mg of dry powder per actuation.

pMDI Delivery

In another aspect, the rapamycin is delivered in the form of aerosolized particles from a pressurized container or dispenser that contains a suitable propellant as described above in connection with propellant-based formulations. In one aspect, the inhaler is a propellant driven inhaler, such as a pMDI device, which releases a metered dose of rapamycin upon each actuation. A typical pMDI device includes a canister containing drug, a drug metering valve, and a mouthpiece. In one aspect of this aspect, the rapamycin is formulated as a suspension in the propellant. In the context of this aspect, the rapamycin is made into a fine powder, which is suspended in the liquefied propellant or propellant blend. The suspension is then stored in a sealed canister under sufficient pressure to maintain the propellant in liquid form. In another aspect, the rapamycin is formulated as a solution. In the context of this aspect, the rapamycin is solubilized in the liquefied propellant or propellant blend. In one aspect, the formulation further includes a stabilizer in an amount suitable to stabilize the formulation against settling, creaming or flocculation for a time sufficient to allow reproducible dosing of the rapamycin after agitation of the formulation. The stabilizer may be present in excess in an amount of about 10 parts by weight to about 5000 parts by weight based on one million parts by total weight of the aerosol formulation. In one aspect, the fluid carrier is 1,1,1,2-tetrafluoroethane, 1,1,1,2,3,3,3-heptafluoropropane or a mixture thereof. In another aspect, the fluid carrier is a hydrocarbon (e.g., n-butane, propane, isopentane, or a mixture thereof). The composition may further include a co-solvent (e.g., ethanol or other suitable co-solvent).

In some aspects, the method further includes administering one or more additional therapeutic agents to the subject.

### Oral Delivery

In some aspects, the composition or product is formulated for oral delivery. In some aspects, the rapamycin is present at about 0.1% to 90% w/w. In some aspects, the rapamycin is present at about 0.25% to 55% w/w, or about 10 to 25% w/w based upon the total weight of the composition. In some aspects, the treprostinil is present at about 0.01% to 90% w/w. In some aspects, the treprostinil is present at about 0.25% to 55% w/w, or about 10 to 25% w/w based upon the total weight of the composition.

In some aspects, the rapamycin has an isomeric B: C ratio of greater than 35:1. In some aspects, the rapamycin has an isomeric B: C ratio of 3.5% to 10%.

In some aspects, the composition further includes a carrier selected from the group consisting of a binder, a lubricant, a preservative, an anti-oxidant, a disintegrating agent, a thickener, a buffer, a surfactant, a suspending agent, an emulsifying agent, a solvent, a stabilizer, or a bioavailability modifier.

Bioavailability modifiers can include surfactants, pH modifiers, P-glycoprotein inhibitors, enzyme inhibitors, and absorption enhancers. In some aspects, a P-glycoprotein inhibitor is selected from the group consisting of vitamin E, polyethylene glycol, poloxamer, a polyoxyethylene castor oil derivative, chrysin, dihydroquercetin, naringenin, diosmin, quercetin, cyclosporin A, verapamil, tamoxifen, quinidine, or a phenothiazine.

In some aspects, the rapamycin, treprostinil, or a combination thereof persists for at least 12 or 24 hours after delivery

### Additives

The compositions of the disclosure may contain one or more additives in addition to any carrier or diluent (such as lactose or mannitol) that is present in the formulation. In one aspect, the one or more additives include one or more surfactants. Surfactants typically have one or more long aliphatic chains, such as fatty acids, which enable them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the disclosure are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides. Each of these is described in more detail below.

### PEG Fatty Acid Esters

Although polyethylene glycol (PEG) itself does not function as a surfactant, a variety of PEG-fatty acid esters have useful surfactant properties. Among the PEG-fatty acid monoesters, esters of lauric acid, oleic acid, and stearic acid are most useful in aspects of the present disclosure. Preferred hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. The HLB values are in the range of 4-20.

Polyethylene glycol fatty acid diesters are also suitable for use as surfactants in the compositions of aspects of the present disclosure. Most preferred hydrophilic surfactants include PEG-20 dilaurate, PEG-20 dioleate, PEG-20 distearate, PEG-32 dilaurate and PEG-32 dioleate. The HLB values are in the range of 5-15.

In general, mixtures of surfactants are also useful in aspects of the present disclosure, including mixtures of two or more commercial surfactants as well as mixtures of surfactants with another additive or additives. Several PEG-fatty acid esters are marketed commercially as mixtures or mono- and diesters.

### Polyethylene Glycol Glycerol Fatty Acid Esters

Preferred hydrophilic surfactants are PEG-20 glyceryl laurate, PEG-30 glyceryl laurate, PEG-40 glyceryl laurate, PEG-20 glyceryl oleate, and PEG-30 glyceryl oleate.

### Alcohol-Oil Transesterification Products

A large number of surfactants of different degrees of hydrophobicity or hydrophilicity can be prepared by reaction of alcohols or polyalcohol with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil trans esterified surfactants, preferred hydrophilic surfactants are PEG-35 castor oil (Incrocas-35), PEG-40 hydrogenated castor oil (Cremophor RH 40), PEG-25 trioleate (TAGAT™ TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol), and PEG-6 caprylic/capric glycerides (Softigen 767). Preferred hydrophobic surfactants in this class include PEG-5 hydrogenated castor oil, PEG-7 hydrogenated castor oil, PEG-9 hydrogenated castor oil, PEG-6 com oil (Labrafil™ M 2125 CS), PEG-6 almond oil (Labrafil™ M 1966 CS), PEG-6 apricot kernel oil (Labrafil™ M 1944 CS), PEG-6 olive oil (Labrafil™ M 1980 CS), PEG-6 peanut oil (Labrafil™ M 1969 CS), PEG-6 hydrogenated palm kernel oil (Labrafil™ M 2130 BS), PEG-6 palm kernel oil (Labrafil™ M 2130 CS), PEG-6 triolein (Labrafil™ b M 2735 CS), PEG-8 com oil (Labrafil™ WL 2609 BS), PEG-20 com glycerides (Crovol M40), and PEG-20 almond glycerides (Crovol A40).

### Polyglyceryl Fatty Acids

Polyglycerol esters of fatty acids are also suitable surfactants for use in aspects of the present disclosure. Among the polyglyceryl fatty acid esters, preferred hydrophobic surfactants include polyglyceryl oleate (Plurol Oleique), polyglyceryl-2 dioleate (Nikkol DGDO), polyglyceryl-10 trioleate, polyglyceryl stearate, polyglyceryl laurate, polyglyceryl myristate, polyglyceryl palmitate, and polyglyceryl linoleate. Preferred hydrophilic surfactants include polyglyceryl-10 laurate (Nikkol Decaglyn 1-L), polyglyceryl-10 oleate (Nikkol Decaglyn 1-0), and polyglyceryl-10 mono, dioleate (Caprol™ PEG 860), polyglyceryl-10 stearate, polyglyceryl-10 laurate, polyglyceryl-10 myristate, polyglyceryl-10 palmitate, polyglyceryl-10 linoleate, polyglyceryl-6 stearate, polyglyceryl-6 laurate, polyglyceryl-6 myristate, polyglyceryl-6 palmitate, and polyglyceryl-6 linoleate. Polyglyceryl polyricinoleates (Polymuls) are also preferred surfactants.

### Propylene Glycol Fatty Acid Esters

Esters of propylene glycol and fatty acids are suitable surfactants for use in aspects of the present disclosure. In this surfactant class, preferred hydrophobic surfactants include propylene glycol monolaurate (Lauroglycol FCC), propylene glycol ricinoleate (Propymuls), propylene glycol monooleate (Myverol P-06), propylene glycol di capryl ate/di caprate (Captex™ 200), and propylene glycol dioctanoate (Captex™ 800).

### Sterol and Sterol Derivatives

Sterols and derivatives of sterols are suitable surfactants for use in aspects of the present disclosure. Preferred derivatives include the polyethylene glycol derivatives. A preferred surfactant in this class is PEG-24 cholesterol ether (Solulan C-24).

### Polyethylene Glycol Sorbitan Fatty Acid Esters

A variety of PEG-sorbitan fatty acid esters are available and are suitable for use as surfactants in aspects of the present disclosure. Among the PEG-sorbitan fatty acid esters, preferred surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80).

### Polyethylene Glycol Alkyl Ethers

Ethers of polyethylene glycol and alkyl alcohols are suitable surfactants for use in aspects of the present disclosure. Preferred ethers include PEG-3 oleyl ether (Volpo 3) and PEG-4 lauryl ether (Brij 30).

### Sugar and its Derivatives

Sugar derivatives are suitable surfactants for use in aspects of the present disclosure. Preferred surfactants in this class include sucrose monopalmitate, sucrose monolaurate, decanoyl-N-methylglucamide, n-decyl-β-D-glucopyranoside, n-decyl-β-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, and octyl-β-D-thioglucopyranoside.

### Polyethylene Glycol Alkyl Phenols

Several PEG-alkyl phenol surfactants are available, such as PEG-10-100 nonyl phenol and PEG-15-100 octyl phenol ether, Tyloxapol, octoxynol, nonoxynol, and are suitable for use in aspects of the present disclosure.

### Polyoxyethylene-Polyoxypropylene (POE-POP) Block Copolymers

The POE-POP block copolymers are a unique class of polymeric surfactants. The unique structure of the surfactants, with hydrophilic POE and hydrophobic POP moieties in well-defined ratios and positions, provides a wide variety of surfactants suitable for use in aspects of the present disclosure. These surfactants are available under various trade names, including Synperonic PE series (ICI); Pluronic™ series (BASF), Emkalyx, Lutrol (BASF), Supronic, Monolan, Pluracare, and Plurodac. The generic term for these polymers is “poloxamer” (CAS 9003 Nov. 6). These polymers have the formula: HO (C2H40) a (C3H60) b (C2H40) aH where “a” and “b” denote the number of polyoxyethylene and polyoxypropylene units, respectively.

Preferred hydrophilic surfactants of this class include Poloxamers 108, 188, 217, 238, 288, 338, and 407. Preferred hydrophobic surfactants in this class include Poloxamers 124, 182, 183, 212, 331, and 335.

### Sorbitan Fatty Acid Esters

Sorbitan esters of fatty acids are suitable surfactants for use in aspects of the present disclosure. Among these esters, preferred hydrophobic surfactants include sorbitan monolaurate (Arlacel 20), sorbitan monopalmitate (Span-40), sorbitan monooleate (Span-80), sorbitan monostearate.

The sorbitan monopalmitate, an amphiphilic derivative of Vitamin C (which has Vitamin C activity), can serve two important functions in solubilization systems. First, it possesses effective polar groups that can modulate the microenvironment. These polar groups are the same groups that make vitamin C itself (ascorbic acid) one of the most water-soluble organic solid compounds available: ascorbic acid is soluble to about 30 wt/wt % in water (very close to the solubility of sodium chloride, for example). And second, when the pH increases so as to convert a fraction of the ascorbyl palmitate to a more soluble salt, such as sodium ascorbyl palmitate.

### Ionic Surfactants

Ionic surfactants, including cationic, anionic and zwitterionic surfactants, are suitable hydrophilic surfactants for use in aspects of the present disclosure. Preferred ionic surfactants include quaternary ammonium salts, fatty acid salts, and bile salts. Specifically, preferred ionic surfactants include benzalkonium chloride, benzethonium chloride, cetylpyridinium chloride, docecyl trimethyl ammonium bromide, sodium docecylsulfates, dialkyl methylbenzyl ammonium chloride, edrophonium chloride, domiphen bromide, dialkylesters of sodium sulfonsuccinic acid, sodium dioctyl sulfosuccinate, sodium cholate, and sodium taurocholate. These quaternary ammonium salts are preferred additives. They can be dissolved in both organic solvents (such as ethanol, acetone, and toluene) and water. This is especially useful for medical device coatings because it simplifies the preparation and coating process and has good adhesive properties. Water insoluble drugs are commonly dissolved in organic solvents.

### Fat-Soluble Vitamins and Salts Thereof

Vitamins A, D, E and K in many of their various forms and provitamin forms are considered as fat-soluble vitamins and in addition to these a number of other vitamins and vitamin sources or close relatives are also fat-soluble and have polar groups, and relatively high octanol-water partition coefficients. Clearly, the general class of such compounds has a history of safe use and high benefit to risk ratio, making them useful as additives in aspects of the present disclosure.

The following examples of fat-soluble vitamin derivatives and/or sources are also useful as additives: Alpha-tocopherol, beta-tocopherol, gamma-tocopherol, delta-tocopherol, tocopherol acetate, ergosterol, 1-alpha-hydroxycholecal-ciferol, vitamin D2, vitamin D3, alpha-carotene, beta-carotene, gamma-carotene, vitamin A, fursultiamine, methylolriboflavin, octotiamine, prosultiamine, riboflavine, vintiamol, dihydrovitamin K1, menadiol diacetate, menadiol dibutyrate, menadiol disulfate, menadiol, vitamin K1, vitamin K1 oxide, vitamins K2, and vitamin K—S(II). Folic acid is also of this type, and although it is water-soluble at physiological pH, it can be formulated in the free acid form. Other derivatives of fat-soluble vitamins useful in aspects of the present invention may easily be obtained via well-known chemical reactions with hydrophilic molecules.

Water-Soluble Vitamins and their Amphiphilic Derivatives

Vitamins B, C, U, pantothenic acid, folic acid, and some of the menadione-related vitamins/provitamins in many of their various forms are considered water-soluble vitamins. These may also be conjugated or complexed with hydrophobic moieties or multivalent ions into amphiphilic forms having relatively high octanol-water partition coefficients and polar groups. Again, such compounds can be of low toxicity and high benefit to risk ratio, making them useful as additives in aspects of the present invention. Salts of these can also be useful as additives in the present disclosure. Examples of water-soluble vitamins and derivatives include, without limitation, acetiamine, benfotiamine, pantothenic acid, cetotiamine, cyclothiamine, dexpanthenol, niacinamide, nicotinic acid, pyridoxal 5-phosphate, nicotinamide ascorbate, riboflavin, riboflavin phosphate, thiamine, folic acid, menadiol diphosphate, menadione sodium bisulfite, menadoxime, vitamin B12, vitamin K5, vitamin K6, vitamin K6, and vitamin U. Also, as mentioned above, folic acid is over a wide pH range including physiological pH, water-soluble, as a salt.

Compounds in which an amino or other basic group is present can easily be modified by simple acid-base reaction with a hydrophobic group-containing acid such as a fatty acid (especially lauric, oleic, myristic, palmitic, stearic, or 2-ethylhexanoic acid), low-solubility amino acid, benzoic acid, salicylic acid, or an acidic fat-soluble vitamin (such as riboflavin). Other compounds might be obtained by reacting to such an acid with another group on the vitamin such as a hydroxyl group to form a linkage such as an ester linkage, etc. Derivatives of a water-soluble vitamin containing an acidic group can be generated in reactions with a hydrophobic group-containing reactant such as stearylamine or riboflavin, for example, to create a compound that is useful in aspects of the present invention. The linkage of a palmitate chain to vitamin C yields ascorbyl palmitate.

### Amino Acids and Their Salts

Alanine, arginine, asparagines, aspartic acid, cysteine, cystine, glutamic acid, glutamine, glycine, histidine, proline, isoleucine, leucine, lysine, methionine, phenylalanine, serine, threonine, tryptophan, tyrosine, valine, and their derivatives are other useful additives in aspects of the disclosure.

Certain amino acids, in their zwitterionic form and/or in a salt form with a monovalent or multivalent ion, have polar groups, relatively high octanol-water partition coefficients, and are useful in aspects of the present invention. In the context of the present disclosure, we take “low-solubility amino acid” to mean an amino acid which has solubility in unbuffered water of less than about 4% (40 mg/mL). These include cystine, tyrosine, tryptophan, leucine, isoleucine, phenylalanine, asparagine, aspartic acid, glutamic acid, and methionine.

### Organic Acids and Their Esters and Anhydrides

Examples are acetic acid and anhydride, benzoic acid and anhydride, acetylsalicylic acid, diflunisal, 2-hydroxyethyl salicylate, diethylenetriaminepentaacetic acid dianhydride, ethylenediaminetetraacetic dianhydride, maleic acid and anhydride, succinic acid and anhydride, diglycolic anhydride, glutaric anhydride, ascorbic acid, citric acid, tartaric acid, lactic acid, oxalic acid aspartic acid, nicotinic acid, 2-pyrrolidone-5-carboxylic acid, and 2-pyrrolidone.

These esters and anhydrides are soluble in organic solvents such as ethanol, acetone, methyl ethyl ketone, ethyl acetate. The water insoluble drugs can be dissolved in organic solvent with these esters and anhydrides, then coated easily on to the medical device, then hydrolyzed under high pH conditions. The hydrolyzed anhydrides or esters are acids or alcohols, which are water soluble and can effectively carry the drugs off the device into the vessel walls.

Examples of inhalable or oral formulations and compositions of rapamycin and Treprostinil contemplated for the present application are herein incorporated by reference in their entirety and include but are not limited to:
U.S. Pat. No. 11,744,797/U.S. Pat. No. 10,307,370/U.S. Pat. No. 11,123,289/U.S. Pat. No. 11,744,797/U.S. Pat. No. 12,171,874/U.S. Pat. No. 11,103,449/U.S. Pat. No. 11,648,199 (LAM Therapeutics/Orphai) US2021/0077504 (Liquidia); U.S. Pat. No. 10,716,793 (United Therapeutics); U.S. Pat. No. 9,604,901 (United Therapeutics); Tyvaso DPI (United Therapeutics)/Yutrepia (Liquidia)/INS1009 (TPIP/Insmed) labels (www.fda.gov/drugsatfda).

TABLE 6
Illustrative Patents and Applications
Incorporated herein by Reference

| Company | Patent Number | Title |
|---|---|---|
| Insmed | US20180153847 | Treprostinil |
| | | Prodrugs |
| Liquidia | US20210077504A1 | Dry Powder |
| | | Treprostinil for |
| | | Pulmonary |
| | | Hypertension |
| Liquidia | U.S. Pat. No. 10,716,793 | Treprostinil |
| | | Administration by |
| | | Inhalation |
| United | U.S. Pat. No. 12,173,021 | Treprostinil |
| Therapeutics | | Prodrugs |
| United | U.S. Pat. No. 12,168,071 | Treprostinil |
| Therapeutics | | Derivatives and Their |
| | | Use in Pharmaceutical |
| | | Compositions |
| United | US20250049742 | Treatment for Interstitial |
| Therapeutics | | Lung Disease |

The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

The disclosure is further described in the following examples, which do not limit the scope of the invention described in the claims.

### Example 1

### In Vitro Pharmacology of Treprostinil and Rapamycin

The in vitro pharmacology plan and methods will involve three distinct studies. The first study will aim to establish an appropriate dose-response (EC25) in human pulmonary artery smooth muscle cells (hPASMCs) and human pulmonary artery endothelial cells (hPAECs) with treprostinil. This will be achieved by using predetermined pharmacodynamic endpoints, including cell proliferation, changes in ID1 expression, and TGFβ1 secretion, through the alteration of intracellular CAMP levels and phosphorylation of S6 kinase.

The second study will focus on performing combination treatments of sub-maximal treprostinil and sub-optimal rapamycin (EC25 or EC50). The goal is to determine the synergistic effects between the two therapeutic modalities on the previously described endpoints.

The third study will aim to verify the contribution of the treprostinil/prostacyclin pathway in the combination condition with rapamycin. This will be assessed by examining the above-described endpoints in the presence of a cAMP/PKA inhibitor (H-89).

TABLE 7
Study Designs

| Study | Description | Model | Endpoints |
|---|---|---|---|
| Study 1 | Vehicle Control(s) | PASMCs + hypoxia | [symbol] cAMP levels |
| | Vehicle Control(s) | PAECs + hypoxia | [symbol] pS6 levels |
| | Trep Dose Response (1 nM-100 uM) | PASMCs + hypoxia | |
| | Trep Dose Response (1 nM-100 uM) | PAECs + hypoxia | |
| Study 2 | Vehicle Control(s) | PASMCs + hypoxia | └ Proliferation |
| | Rap Alone Low - EC25 (0.2) | | ┌ ID1-expression |
| | Rap Alone Mid - EC50 (0.75 nM) | | └ cAMP levels |
| | Trep Alone - EC25 (TBD) | | └ pS6 levels |
| | Trep EC25/RAP Low Combo | | ┌ TGFβ1 levels |
| | Trep EC25/RAP Mid Combo | | |
| | Vehicle Control(s) | PAECs + hypoxia | |
| | Rap Alone Low - EC25 (~0.5 nM) | | |
| | Rap Alone Mid - EC50 (1.5 nM) | | |
| | Trep Alone - EC25 (TBD) | | |
| | Trep EC25/RAP Low Combo | | |
| | Trep EC25/RAP Mid Combo | | |
| Study 3 | Vehicle Control(s) | PASMCs +/− hypoxia | └ Proliferation |
| | Trep Alone - EC25 (TBD) | | └ ID1-expression |
| | Trep EC25/RAP Low Combo | | └ cAMP levels |
| | Trep EC25/RAP Mid Combo | | └ pS6 levels |
| | Trep Alone - EC25 + H89 | | ┌ TGFβ1 levels |
| | Trep EC25/RAP Low Combo + H89 | | |
| | Trep EC25/RAP Mid Combo + H89 | | |
| | Vehicle Control(s) | PAECs +/− hypoxia | |
| | Trep Alone - EC25 (TBD) | | |
| | Trep EC25/RAP Low Combo | | |
| | Trep EC25/RAP Mid Combo | | |
| | Trep Alone - EC25 + H89 | | |
| | Trep EC25/RAP Low Combo + H89 | | |
| | Trep EC25/RAP Mid Combo + H89 | | |

### Cells, Growth Media, and Test Article

The study will utilize Human Pulmonary Artery Smooth Muscle Cells (hPASMC) and Human Pulmonary Artery Endothelial Cells (hPAEC), both supplied by ScienCell Research Laboratories. The hPASMCs, catalog number 3110, are stored in liquid nitrogen, as were the hPAECs, catalog number 3100. For growth media, Smooth Muscle Cell Medium, also from ScienCell Research Laboratories with catalog number 1101, are used and stored at 2±4° C. Similarly, Endothelial Cell Medium from the same supplier, catalog number 1001, re stored under the same conditions.

The test articles include rapamycin (sirolimus) from Adooq Bioscience, catalog number A10782, stored at −20° C., and treprostinil from Sigma-Aldrich, catalog number SML1531, also stored at −20° C. Additionally, H-89, dihydrochloride, another test article from Sigma-Aldrich with catalog number 371963-M, is stored at 2-8° C.

### Methods

Primary hPASMCs will be put in culture and grown in smooth muscle cell medium supplemented with 2% FBS, 1% smooth muscle cell growth supplement and 1% penicillin/streptomycin. Primary hPAECs will be put in culture and grown in endothelial medium, 5% FBS, 1% endothelial cell growth supplement and 1% penicillin/streptomycin. hPASMCs and hPAECs between passages 3 and 15 will be plated in 12-well plates (for proliferation assays, TGFβ1 and qPCR assays) and 96-well plates (for pS6 and cAMP assessment) at ~1×10⁵ cells/cm² and allowed to adhere overnight, in triplicates for each treatment condition. Cells will either be kept in normoxia (20.9% O₂, 5% CO₂) or under chronic hypoxia (1% O₂, 5% CO₂) for 7 days.

For proliferation assessments, TGFβ1 and ID1 qPCR: Seven-days after plating in the presence of normoxia or hypoxic conditions, cells will be treated with vehicle control (0 nM) or treprostinil (range covering 1 nM to 100 μM) and/or rapamycin (EC25 range of 0.1-0.5 nM; EC50 range of 1-2 nM) for 6, 24, 28 or 72 additional hours. At each timepoint, culture media will be retained from each condition/replicate for assessment of TGFβ1 levels (R&D Systems, Catalog #DB100C or similar) and cells will be counted with Trypan Blue (1:1) using a TC20TM Automated Cell Counter (BioRad). The remaining cells from each replicate will be pooled and centrifuged at 200 g for 5 min at 4° C., washed with cold PBS, centrifuged at 2,000 g for 5 min at 4° C., and processed for RNA isolation using TRIzol (Invitrogen #15596026). Briefly, the cell pellet will be lysed with TRIZOL™ Reagent, chloroform will be added, and the aqueous phase will be isolated by centrifugation. The RNA will then be precipitated with isopropanol and centrifugation, washed with ethanol, and resuspended in water. A fixed amount of mRNA (0.01-1 μg) will then be reverse transcribed by qScript cDNA Synthesis (QuantaBio #95047). The cDNA of human ID1 and human β-actin (reference control) will then be amplified and the amplicon will be quantified by PERFECTA® SYBR® Green (QuantaBio #95073). The primer sequences are listed in Table 9. Absolute cell counts and concentration of TGFβ1levels (pg/mL) will be reported. Relative expression of ID1 to β-actin will be expressed using the delta-delta Ct method (2^{-ΔΔCt}).

For S6 phosphorylation: Using the 96-well plates, after 7 days of hypoxic or normoxic conditions, cells will be serum starved for 60 mins prior to the addition of treprostinil (range covering 1 nM to 100 μM) or vehicle control (0 nM). After 60 min of treatment, cells will be processed for quantification of phosphorylation of Ribosomal Protein S6 (RPS6, Uniprot P62753) using Thunder™ Phospho-RPS6 (S240/S244) TR-FRET signaling assay (Bioauxilium #P70S6KP). Data will be reported as an HTRF ratio of the donor/acceptor in the assay and compared to the untreated conditions (Vehicle control).

For cAMP and evaluation: Cells from a 96-well plate will be processed for CAMP levels using the CAMP-GLO™ Assay (Promega, V1501) following the manufacture recommended procedures. Fold change in cAMP levels versus vehicle control conditions will be determined.

TABLE 9
QPCR Primer Sequences

| | Gene | | |
|---|---|---|---|
| Gene | Accession | | |
| Name | ID | Forward Primer | Reverse Primer |
| Human | NM_181353 | ctgctctacgacat | tgacgtgctggaga |
| ID1 | | gaacggc | atctcca |
| | | (SEQ ID NO: 1) | (SEQ ID NO: 2) |
| Human | EF095209 | gcaccacaccttct | gtcatcttctcgcg |
| β-actin | | acaatga | gttggc |
| | | (SEQ ID NO: 3) | (SEQ ID NO: 4) |

Study 1: Treprostinil treatment alone is expected to attenuate hypoxia-induced proliferation in hPASMCs by elevating ID1 (a target of the BMP-signaling cascade), which interferes with the cell cycle progression of VSMCs. Intracellularly, this occurs through an increase in intracellular cAMP (via the PKA pathway) and/or the inhibition of S6-kinase phosphorylation (through the mTOR pathway). In hPAECs, the proliferation effects are less pronounced, as these cells do not typically hyper-proliferate in response to hypoxia. However, they respond to treprostinil in terms of ID1 expression, which helps maintain endothelial cell quiescence and stability. Regarding TGFβ1 secretion, which is associated with inducing fibrotic changes in the diseased state, the origin within the pulmonary vasculature is multifaceted and can arise from both cell types. In general, changes under normoxic conditions are expected to be less pronounced and serve as experimental controls and comparators.

Study 2: In the combination condition with rapamycin, a significant reduction in the concentration of treprostinil (EC25) should be required to attenuate proliferation, increase ID1 expression, and reduce TGFβ1 secretion—key markers of a transition from a diseased to a normal state. The magnitude of these effects may also be altered in the combination scenario where neither rapamycin or treprostinil have a maximal effect alone, showing the potential for improved clinical outcomes when these agents are combined.

Study 3: This paradigm is included to demonstrate the contribution of the primary target of the prostacyclin receptor activity in eliciting changes in the function outcomes in the two cell types. The inhibition of cAMP generation pharmacologically with H-89 should reduce the effects seen in a combination scenario with rapamycin. Again, this demonstrates the potential need for both agents to observe maximal effectiveness in these in vitro models of vascular cell dysfunction.

### Example 2

### Combination Therapy with Inhaled Rapamycin and Oral Treprostinil

In this study, the effects of combining inhaled rapamycin with oral treprostinil for the treatment of pulmonary hypertension (PH) will be evaluated.

Methods: Patients with confirmed PH are randomized into four treatment groups: (1) Inhaled rapamycin alone, (2) Oral treprostinil alone, (3) Combination of inhaled rapamycin and oral treprostinil, and (4) Placebo.

Inhaled rapamycin is administered at a dose of 100 ug once daily using a dry powder inhaler. Oral treprostinil is administered at a dose of 0.25 mg twice daily, titrated up to 1 mg twice daily as tolerated. The combination group receives both treatments at the same doses. Treatment will continue for 24 weeks.

Endpoints include changes in 6-minute walk distance (6MWD), pulmonary vascular resistance (PVR), mean pulmonary arterial pressure (mPAP), N-terminal pro-brain natriuretic peptide (NT-proBNP) levels, and World Health Organization (WHO) functional class.

Results: After 24 weeks of treatment, the combination therapy group is expected to show greater improvements compared to either monotherapy group. The combination therapy demonstrates remarkable synergistic effects across all measured endpoints. The results will show that the combination of rapamycin and treprostinil provides benefits beyond what would be expected from simply adding the effects of the individual therapies.

The combination therapy is expected to be well-tolerated, with no unexpected adverse events beyond those typically associated with each individual drug. The inhaled delivery of rapamycin will result in lower systemic exposure and reduced side effects compared to oral administration.

This study will demonstrate that the combination of inhaled rapamycin and oral treprostinil provides synergistic benefits in the treatment of pulmonary hypertension, offering a novel therapeutic approach for patients with this condition. The combination therapy will lead to greater improvements in exercise capacity, hemodynamics, and functional status compared to either treatment alone.

### Example 3

### Early Phase 2 Trial of Rapamycin in Group 1 and 3 pH

In one illustrative example, 10 subjects with pulmonary hypertension (PH), Group 1 or Group 3, were administered inhaled DPI rapamycin on a daily basis on top of standard of care PH therapies, including inhaled, oral, and subcutaneous treprostinil.

As an enrollment inclusion criteria, subjects had WHO Functional Class III at baseline were entered into the study. Subsequent to the addition of inhaled rapamycin, 50% of subjects improved their WHO Functional Class from III to II, including both PH Group 1 and Group 3 subjects. Subjects improved to Functional Class II as early as one week after initiating treatment, and no subjects deteriorated to Functional Class III after achieving Functional Class II, over treatment periods as long as 24 weeks.

Further, subjects were measured on their 6MWD before treatment and after 16 and 24 weeks of treatment. At 16 and 24 weeks, improvements in 6MWD were observed in a majority of subjects, with improvements between 22 meters and 183 meters (11-46%).

Subjects were also tested for pulmonary function, and levels of NT-pro-BNP on a periodic basis. FVC improvements were observed, in some cases as early as 1 week subsequent to initiation of treatment, in both Group 1 and Group 3 PH subjects, with improvements of as much as 119% (2-119%). DLCO measurements were similarly observed to improve as early as 1 week after the initiation of treatment, with improvements as much as 4%-13%. NT-proBNP levels were measured at baseline and at 24 weeks, with declines observed in several subjects, with declines as much as 12%-47% observed.

All of the above points refer only to subjects who had treprostinil as SOC medication and only covers the current data in an ongoing, open-label clinical trial.

KEY:
FVC—forced vital capacity, see:
my.clevelandclinic.org/health/diagnostics/17833-spirometry
DLCO—diffusing capacity for carbon monoxide, see:
my.clevelandclinic.org/health/diagnostics/15445-dlco
6MWD—6 minute walk distance
NT Pro-BNP—N-terminal pro b-type natriuretic peptide, see:
medlineplus.gov/lab-tests/natriuretic-peptide-tests-bnp-nt-probnp/

### Example 4

### In Vitro Pharmacology of Treprostinil and Rapamycin

This study was conducted to characterize the anti-proliferative effects of treprostinil and rapamycin alone and in combination following platelet-derived growth factor (PDGF)-induced proliferation of human pulmonary artery smooth muscle cells (hPASMC). PDGF was used as the proliferative stimulus because it is a well-established mitogen for PASMCs and a key driver of pulmonary vascular remodeling in pulmonary hypertension, where elevated PDGF signaling contributes to smooth muscle hyperplasia and vascular thickening. In isolation, both rapamycin and treprostinil are known to be able to prevent the proliferation induced by PDGF.

### Methods

### In Vitro PDGF-Stimulation of Human PASMCs

Primary human PASMCs from healthy donors were cultured in Smooth Muscle Cell Medium supplemented with 2% FBS, 1% smooth muscle cell growth supplement, and 1% penicillin/streptomycin. All culture vessels were coated with gelatin prior to use, and cells between passages 3 and 15 were seeded into 12-well plates at approximately 10,000 cells/cm². After overnight adherence, cells were switched to serum-free medium containing growth supplement and starved for 24 hours before treatment. Reagents included recombinant human PDGF-BB, Rapamycin, and Treprostinil.

PDGF-BB was prepared in sterile water to generate a 100 μg/mL stock and diluting this to a 1 ug/mL working solution in 0.1% BSA before aliquoting and storing at −20° C.; on the day of use, this working stock was further diluted in serum-free media to obtain final concentrations. Treprostinil and rapamycin were prepared as 10 mM stock solutions in 100% DMSO and diluted in serum-free media to generate the required concentrations. The vehicle control condition contained a final DMSO concentration at the highest concentration present in any treatment (e.g., the 0.25 nM Rapamycin and 10 nM treprostinil condition).

The concentration of PDGF required to induce at least a 50% increase in proliferation relative to untreated cells for these particular lots of PSAMCs was determined to be 25 ng/mL across all donor cell lines. To assess the individual and combined effects of treprostinil and rapamycin on PDGF-induced proliferation, cells were pre-incubated for one hour in serum-free medium containing 10× concentrations of treprostinil (0, 2, 4, 6, 8, or 10 nM), either alone or in combination with rapamycin (0, 0.1, or 0.25 nM). Rapamycin at 0.1 and 0.25 nM alone was also evaluated. These concentrations were selected to be low to subtherapeutic in proliferation assays based upon prior acute cAMP dose-response evaluation for treprostinil (EC₅₀~26 nM) and S6-phosphorylation inhibition response for rapamycin (IC₅₀~0.59 nM) in PSAMCs. PDGF was then added to a final concentration of 25 ng/ml by spiking one-tenth culture volume of a 10×PDGF solution. After 24 hours, the treatment and PDGF-spiking procedure was repeated. At 48 hours, cells were then harvested and manually counted. This assay was repeated three times using independent passages for each donor cell line. The summary of each donor and replicate results reported as percent of vehicle control can be found in, 11 and 12.

TABLE 10
Summary of Vehicle Control, PDGF Treprostinil Alone Results

| | | PDGF (ng/mL) | | | | | | |
|---|---|---|---|---|---|---|---|---|
| | | 0 | 25 | 25 | 25 | 25 | 25 | 25 |
| | | Treprostinil (nM) | | | | | | |
| | | 0 | 0 | 2 | 4 | 6 | 8 | 10 |
| | | Rapamycin (nM) | | | | | | |
| | | 0 | 0 | 0 | 0 | 0 | 0 | 0 |
| Donor 1 | Replicate | 100 | 218.9 | 222.2 | 179.0 | 172.8 | 137 | 130.9 |
| | 1 | | | | | | | |
| Donor 1 | Replicate | 100 | 159.8 | 148 | 138.2 | 122.5 | 157.8 | 118.6 |
| | 2 | | | | | | | |
| Donor 1 | Replicate | 100 | 256.8 | 244.3 | 189.6 | 223.8 | 202.3 | 185.7 |
| | 3 | | | | | | | |
| Donor 2 | Replicate | 100 | 218.8 | 258.8 | 169.5 | 134.5 | 188.7 | 137.9 |
| | 1 | | | | | | | |
| Donor 2 | Replicate | 100 | 237.7 | 221.2 | 158.6 | 217.2 | 254.5 | 245.5 |
| | 2 | | | | | | | |
| Donor 2 | Replicate | 100 | 188.7 | 170.5 | 110.5 | 132.6 | 116.8 | 56.84 |
| | 3 | | | | | | | |
| Donor 3 | Replicate | 100 | 231 | 269.6 | 115.7 | 125.5 | 188.2 | 131.4 |
| | 1 | | | | | | | |
| Donor 3 | Replicate | 100 | 179.3 | 170.5 | 158.9 | 137.9 | 136.8 | 116.8 |
| | 2 | | | | | | | |
| Donor 3 | Replicate | 100 | 203.2 | 165.3 | 150.3 | 127.2 | 144.5 | 116.8 |
| | 3 | | | | | | | |

Summary of each experimental replicate change in proliferation for treprostinil alone versus the Vehicle control conditions (0 ng/mL PDGF, 0 ng/mL Treprostinil and 0 ng/mL rapamycin). For each experiment, a minimum of three technical replicates to achieve a mean response reported here.

TABLE 11
Summary Rapamycin (0.1 nM) and Treprostinil
Combination Results

| | | PDGF (ng/mL) | | | | | |
|---|---|---|---|---|---|---|---|
| | | 25 | 25 | 25 | 25 | 25 | 25 |
| | | Treprostinil (nM) | | | | | |
| | | 0 | 2 | 4 | 6 | 8 | 10 |
| | | Rapamycin (nM) | | | | | |
| | | 0.1 | 0.1 | 0.1 | 0.1 | 0.1 | 0.1 |
| Donor 1 | Replicate 1 | 234.6 | 195.1 | 97.53 | 87.65 | 134.6 | 102.5 |
| Donor 1 | Replicate 2 | 123.5 | 123.5 | 127.5 | 106.9 | 117.6 | 117.6 |
| Donor 1 | Replicate 3 | 199.3 | 130 | 94.79 | 108.5 | 174.9 | 121.2 |
| Donor 2 | Replicate 1 | 136.7 | 142.4 | 134.5 | 154.8 | 102.8 | 100.6 |
| Donor 2 | Replicate 2 | 150.5 | 174.7 | 86.87 | 104 | 184.8 | 153.5 |
| Donor 2 | Replicate 3 | 154.7 | 148.4 | 167.4 | 224.2 | 214.7 | 183.2 |
| Donor 3 | Replicate 1 | 252.9 | 182.4 | 103.9 | 123.5 | 122.5 | 122.5 |
| Donor 3 | Replicate 2 | 126.3 | 123.2 | 106.3 | 129.5 | 141.1 | 113.7 |
| Donor 3 | Replicate 3 | 111.0 | 124.9 | 109.8 | 100.6 | 83.24 | 104 |

Summary of each experimental replicate change in proliferation for the combination of rapamycin (0.1 nM) and treprostinil (0, 2, 4, 6, 8, 10 nM) versus the Vehicle control conditions (0 ng/mL PDGF, 0 ng/mL Treprostinil and 0 ng/mL rapamycin in). For each experiment, a minimum of three technical replicates to achieve a mean response reported here.

TABLE 12
Summary Rapamycin (0.25 nM) and Treprostinil Combination Results

| | | PDGF (ng/mL) | | | | | |
|---|---|---|---|---|---|---|---|
| | | 25 | 25 | 25 | 25 | 25 | 25 |
| | | Treprostinil (nM) | | | | | |
| | | 0 | 2 | 4 | 6 | 8 | 10 |
| | | Rapamycin (nM) | | | | | |
| | | 0.25 | 0.25 | 0.25 | 0.25 | 0.25 | 0.25 |
| Donor 1 | Replicate 1 | 108.6 | 180.2 | 117.3 | 96.3 | 125.9 | 121.0 |
| Donor 1 | Replicate 2 | 138.2 | 109.8 | 116.7 | 125.5 | 122.5 | 106.9 |
| Donor 1 | Replicate 3 | 156.4 | 151.5 | 102.6 | 100.7 | 116.3 | 99.67 |
| Donor 2 | Replicate 1 | 207.9 | 171.8 | 114.1 | 92.66 | 123.2 | 91.53 |
| Donor 2 | Replicate 2 | 208.1 | 191.9 | 127.3 | 101 | 106.1 | 135.4 |
| Donor 2 | Replicate 3 | 97.89 | 78.95 | 50.53 | 66.32 | 88.42 | 82.11 |
| Donor 3 | Replicate 1 | 128.4 | 97.06 | 100.0 | 130.4 | 135.3 | 152.9 |
| Donor 3 | Replicate 2 | 154.7 | 125.3 | 114.7 | 120.0 | 148.4 | 117.9 |
| Donor 3 | Replicate 3 | 119.1 | 100.6 | 82.08 | 112.1 | 94.8 | 101.7 |

Summary of each experimental replicate change in proliferation for the combination of rapamycin (0.25 nM) and treprostinil (0, 2, 4, 6, 8, 10 nM) versus the Vehicle control conditions (0 ng/mL PDGF, 0 ng/mL Treprostinil and 0 ng/mL rapamycin in). For each experiment, a minimum of three technical replicates to achieve a mean response reported here.

### Highest Single Agent (HSA) Analysis

Highest Single Agent (HSA) analysis was performed to determine whether the combined effect of treprostinil and rapamycin exceeded the maximal effect of either agent alone (Fouquier 2015). For each treatment condition, the observed percent inhibition of the combination was compared to the greater of the two corresponding single-agent inhibitions. The HSA synergy score (S_{HSA}) was calculated for each treatment combination as follows:

I_{obs}=100×((PDGF Alone−Combination Observed)/PDGF Alone)

I_{HSA Ref}=Max (I_{Obs Trep Alone}, I_{Obs Rap Alone})

S_{HSA}=I_{obs}−I_{HSA Ref}

Positive values indicate potential synergy, values around zero indicate additivity, and negative values indicate antagonism.

### Bliss Synergy Analysis

Bliss independence model was used to evaluate synergy by comparing the observed effect of the combination to the effect expected if the two agents act independently. Bliss assumes independent mechanisms and is more permissive than the HSA model, which requires the combination to outperform the best single agent, in defining synergy. Therefore, Bliss will call synergy in cases where HSA still calls the interaction additive. For each condition, the fractional inhibition of treprostinil (I_{A}) and rapamycin (I_{B}) was calculated relative to the PDGF-stimulated control as well as the observed combination effect (I_{obs}).

I_{A,B}=1−((Observed Effect)/PDGF Alone)

I_{obs}=1−((Observed Combination Effect)/PDGF Alone)

The expected Bliss inhibition was then computed as:

I_{Bliss}=I_{A}+I_{B}−(I_{A})(I_{B})

The Bliss Synergy Score (S_{Bliss}) was defined as the percent difference between the observed combination inhibition (I_{obs}) and the Bliss-predicted value (I_{Bliss}), such that:

S_{Bliss}=100*(I_{Obs}−I_{Bliss})

Positive values indicate potential synergy, values around zero indicate additivity, and negative values indicate antagonism.

### Results:

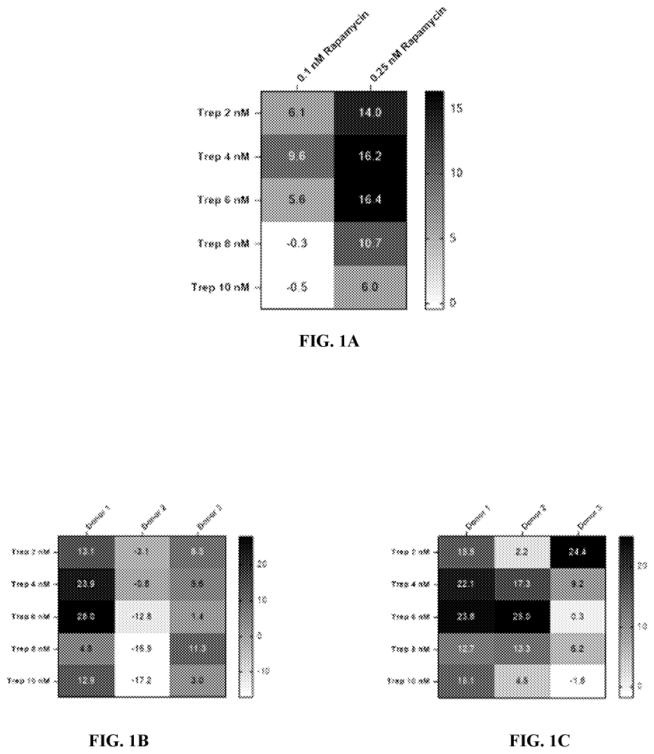

Treprostinil and rapamycin demonstrated dose-dependent interactions in PDGF-stimulated human PSAMCs. HSA analysis showed that treprostinil and rapamycin produced modest but consistent synergy at low-dose combinations, particularly at 2-6 nM treprostinil with 0.25 nM rapamycin (FIG. 1A). These combinations exceeded the inhibitory effect of either agent alone, indicating that the interaction was not solely driven by the stronger monotherapy. At higher treprostinil concentrations (8-10 nM), HSA scores shifted toward additive or mildly antagonistic values, suggesting diminishing benefit from combining the agents at these doses. Donor-level analyses revealed heterogeneity, with some donors exhibiting clear low-dose synergy while others showed predominantly additive responses (FIGS. 1B and 1C). Although any positive HSA score suggests potential synergy, to aid interpretation, HSA scores should be contextualized using a ±10% window, which approximates the inherent variability of cell-based proliferation assays; therefore, values greater than 10 were considered strong and biologically meaningful synergy, values between −10 and 10 are likely to be considered additive given variability in the cell system, and values below −10 were considered antagonistic. Overall, HSA analysis demonstrates that the combination of treprostinil (2-8 nM) and 0.25 nM rapamycin can clearly outperform the best single agent under several combination conditions and donors and provide a model-free foundation for evaluating whether more mechanistic synergy can be detected using the Bliss independence analysis. These data are consistent with a dose-sparing interaction, supporting the prospective evaluation of whether clinically meaningful reductions in treprostinil and/or rapamycin exposure (e.g., on the order of 10-50% relative to monotherapy targets) can maintain or enhance pharmacodynamic effects in patients with PH.

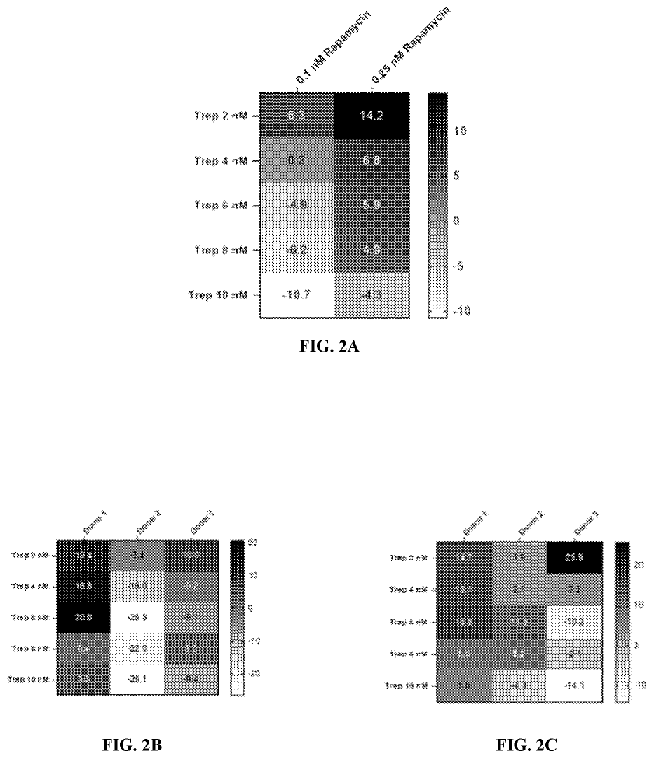

Using Bliss independence analysis, the combination matrix showed low-dose rapamycin (0.1 or 0.25 nM) enhanced the anti-proliferative activity of treprostinil as indicated by positive Bliss Synergy Scores (2A). In particular, 2-6 nM of treprostinil showed consistently positive scores indicating synergy while higher treprostinil concentrations (8-10 nM) showed diminished or negative Bliss Scores, indicating additive or antagonistic interactions. Donor-level analyses revealed expected biological heterogeneity as some donors exhibited strong synergy at low treprostinil doses, whereas others showed predominantly additive or antagonistic responses across the same range (FIG. 2B, 2C). Like the HSA analysis, Bliss scores can be interpreted using a ±10% window to account for assay variability, with values >10 indicating clear synergy, −10 to +10 likely indicating additive given variability in the cell system, and below −10 indicating antagonism. These findings indicate that treprostinil and rapamycin Bliss synergy is most pronounced at 2 nM treprostinil and 0.25 nM rapamycin and with some variability across primary donor cell sources.

Importantly, the rapamycin and treprostinil concentrations used in this study were intentionally selected to be well below the IC₅₀ for rapamycin-induced mTOR inhibition (~0.59 nM) and the EC₅₀ for treprostinil-induced cAMP activation (~26 nM) in these cells, allowing detection of cooperative effects without saturating either pathway. This low-dose strategy highlights that synergy emerges not from maximal rapamycin or treprostinil activity alone, but from complementary pathway modulation. From a translational perspective, the observation that synergy occurs at sub-IC₅₀ of rapamycin and sub-EC₅₀ treprostinil concentrations suggests that combination regimens may achieve meaningful biological effects at doses lower than those required for either agent alone. A similar interaction in vivo, of such low-dose synergy could support combination products designed to reduce individual drug exposure and dosing frequency while maintaining or enhancing efficacy leading to improved tolerability and a wider therapeutic window.

### REFERENCES

Fouquier, J., and M. Guedj. “Analysis of drug combinations: current methodological landscape.” Pharmacol Res Perspect 3.3 (2015): e00149.
Houssaini, Amal, et al. “Rapamycin reverses pulmonary artery smooth muscle cell proliferation in pulmonary hypertension.” American journal of respiratory cell and molecular biology 48.5 (2013): 568-577.
Krymskaya, Vera P., et al. “mTOR is required for pulmonary arterial vascular smooth muscle cell proliferation under chronic hypoxia.” The FASEB Journal 25.6 (2011): 1922.
Lambers, Christopher, et al. “Mechanism of anti-remodelling action of treprostinil in human pulmonary arterial smooth muscle cells.” PLOS One 13.11 (2018): e0205195.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A method of treating pulmonary hypertension (PH) in a subject in need thereof comprising administering to the subject by inhalation:
(i) rapamycin, and
(ii) treprostinil or treprostinil palmitil, thereby treating PH in the subject.

2. The method of claim 1, wherein rapamycin is administered in a total daily dose of about 5 μg to 500 μg.

3. The method of claim 2, wherein rapamycin is administered in a total daily dose of 100 μg or 200 μg.

4. The method of claim 2, wherein rapamycin is administered at least once daily or less than once daily.

5. The method of claim 1, wherein treprostinil is administered in a total daily dose of about 1 μg to 1000 μg.

6. The method of claim 5, wherein treprostinil is administered in a total daily dose of about 10 μg to1000 μg.

7. The method of claim 1, wherein a dose or frequency of administration of treprostinil or treprostinil palmitil administered in combination with rapamycin is lower than a dose or frequency of administration of treprostinil or treprostinil palmitil administered in the absence of rapamycin.

8. The method of claim 1, wherein a dose or frequency of administration of rapamycin administered in combination with treprostinil or treprostinil palmitil is lower than a dose or frequency of administration of rapamycin administered in the absence of treprostinil or treprostinil palmitil.

9. The method of claim 1, further comprising administering one or more additional therapeutic agents to the subject.

10. (canceled)

11. The method of claim 1, wherein the PH is pulmonary arterial hypertension, pulmonary hypertension associated with interstitial lung disease, chronic thromboembolic pulmonary hypertension, sarcoidosis-associated pulmonary hypertension, pulmonary hypertension associated with COPD, idiopathic pulmonary hypertension, pulmonary hypertension due to lung disease and/or hypoxia, or Group 5 PH due to unclear or multifactorial etiology.

12. The method of claim 1, wherein administration of rapamycin and administration of treprostinil or treprostinil palmitil is simultaneous or sequential in either order.

13. The method of claim 1, wherein the rapamycin is administered by inhalation; and the treprostinil or treprostinil palmitil is administered by inhalation, either simultaneously or sequentially in either order.

14-15. (canceled)

16. A method of inhibiting proliferation of a pulmonary artery smooth muscle cells (PASMCs) comprising contacting the PASMCs with (i) rapamycin formulated for inhalation and (ii) treprostinil or treprostinil palmitil formulated for inhalation, thereby inhibiting the proliferation of the PASMCs.

17. A method of inhibiting smooth muscle cell proliferation and/or inhibiting vascular thickening and/or improving pulmonary vascular remodeling in a subject in need thereof, comprising administering to the subject (i) rapamycin, formulated for inhalation, and (ii) treprostinil or treprostinil palmitil, formulated for inhalation, thereby inhibiting smooth muscle cell proliferation and/or inhibiting vascular thickening and/or improving pulmonary vascular remodeling.

18. A pharmaceutical product comprising:
(i) rapamycin, formulated for inhalation; and
(ii) treprostinil, an analog thereof, a prodrug thereof or a derivative thereof, formulated for inhalation.

19-20. (canceled)

21. The product of claim 18, wherein the product comprises about 0.1% to 20% w/w treprostinil and about 0.1% to 20% rapamycin w/w.

22. The product of claim 18, further comprising one or more pharmaceutically acceptable carrier(s), wherein the pharmaceutically acceptable carrier is selected from the group consisting of arabinose, glucose, fructose, ribose, mannose, sucrose, trehalose, lactose, maltose, starches, dextran, mannitol, lysine, leucine, isoleucine, dipalmitoylphosphatidylcholine, lecithin, polylactic acid, poly(lactic-co-glutamic) acid, and xylitol, or mixtures thereof.

23-24. (canceled)

25. The product of claim 18, wherein the rapamycin is present in an amount of about 0.025 mg to 3.0 mg.

26. The product of claim 18, wherein the treprostinil is present in an amount of about 20 μg to 20 mg for inhalation.

27-30. (canceled)

31. The product of claim 18, wherein the treprostinil is a treprostinil analog, pharmaceutically acceptable salt of treprostinil, or a treprostinil prodrug.

32. The product of claim 31, wherein the treprostinil is selected from treprostinil diethanolamine, treprostinil diolamine, treprostinil palmitil, treprostinil phosphate, treprostinil diproprionate, treprostinil diacetate, treprostinil palmitil inhaled powder (TPIP), long chain treprostinil prodrug (C12, C14 or C16 long chain treprostinil prodrug), or treprostinil dibutanoate.

33. The method of claim 1, comprising administering to the subject:
(a) (i) rapamycin, by inhalation, and
(ii) treprostinil, by inhalation, or
(b) (i) rapamycin, by inhalation, and
(ii) treprostinil palmitil, by inhalation.

34. The method of claim 33, wherein the treprostinil palmitil is treprostinil palmitil inhaled powder (TPIP).

35. The method of claim 2, wherein rapamycin is administered in a total daily dose of about 50 μg to 500 μg or about 100 μg to 300 μg.

36. The method of claim 5, wherein treprostinil is administered at a dose of 16, 32, 48, 64, 84, 150, 250 μg, 500, 750, or 1000 ug.

37. The method of claim 5, wherein treprostinil is administered 1, 2, 3, 4 or more times daily.

38. The method of claim 1, wherein a dose or frequency of administration of rapamycin and the dose or frequency of administration of treprostinil or treprostinil palmitil are each lower than a dose or frequency of administration of each agent in the absence of the other.

39. The method of claim 1, wherein the PH is due to an underlying disease selected from idiopathic pulmonary fibrosis, progressive pulmonary fibrosis, right heart failure secondary to pulmonary hypertension, lymphangioleiomyomatosis, COPD, chronic fibrotic lung disease, interstitial lung disease, chronic lung disease, pulmonary capillary hemangiomatosis, hereditary hemorrhagic telangiectasia, idiopathic interstitial pneumonia, pulmonary sarcoidosis, systemic sclerosis associated ILD, fibrotic lung disorders, chronic hypersensitivity pneumonitis (CHP), occupational lung disease (drug or radiation-induced), combined pulmonary fibrosis and emphysema (CPFE), pulmonary hypertension associated with COPD, idiopathic pulmonary hypertension, pulmonary hypertension due to lung disease and/or hypoxia, Group 5 PH due to unclear or multifactorial etiology, hematologic disorders, myeloproliferative disorders, sarcoidosis, Langerhans cell histiocytosis, LAM, neurofibromatosis, vasculitis, metabolic diseases, glycogen storage diseases, Gaucher disease, or thyroid disorders, pulmonary arterial hypertension, pulmonary hypertension associated with interstitial lung disease, chronic thromboembolic pulmonary hypertension, or sarcoidosis-associated pulmonary hypertension.

40. The method of claim 1, wherein the rapamycin is administered by inhalation as a dry powder for pulmonary delivery or nebulized solution; and the treprostinil is administered by inhalation as a dry powder or nebulized solution, either simultaneously or sequentially in either order.

41. The method of claim 1, wherein administering rapamycin and treprostinil comprises administering a single combination formulation.


SEQUENCE LISTING (4 sequences)

SEQ ID NO: 1 - DNA, 21 bp, synthetic construct
  source 1..21: mol_type = other DNA
ctgctctacgacatgaacggc

SEQ ID NO: 2 - DNA, 21 bp, synthetic construct
  source 1..21: mol_type = other DNA
tgacgtgctggagaatctcca

SEQ ID NO: 3 - DNA, 21 bp, synthetic construct
  source 1..21: mol_type = other DNA
gcaccacaccttctacaatga

SEQ ID NO: 4 - DNA, 20 bp, synthetic construct
  source 1..20: mol_type = other DNA
gtcatcttctcgcggttggc